(19) United States
(12) Patent Application Publication
(10) Pub. No.: US 2022/0136990 A1
(43) Pub. Date: May 5, 2022

(54) METHOD AND SENSOR FOR DETERMINING A VALUE INDICATING THE IMPEDANCE OF A SUSPENSION

(71) Applicant: HAMILTON BONADUZ AG, Bonaduz (CH)

(72) Inventors: Valentin VERSCHININ, Chur (CH); Manuel IMHOF, Felsberg (CH)

(21) Appl. No.: 17/297,571

(22) PCT Filed: Nov. 29, 2019
(86) PCT No.: PCT/EP2019/083114

§ 371 Date: Jan. 18, 2022

(30) Foreign Application Priority Data
Nov. 30, 2018 (DE) 10 2018 130 487.0

(51) Int. Cl. G01N 27/02; G01N 33/487

(52) U.S. Cl. CPC G01N 27/028; G01N 33/48735

(57) ABSTRACT

A method for determining a value indicative of the impedance of a suspension in the framework of impedance spectroscopy comprises the following steps: generating an excitation current through the suspension, oscillating at an excitation frequency; determining a first impedance measurement value on the basis of the excitation current and a first voltage at a first pair of measurement electrodes; determining a second impedance measurement value on the basis of the excitation current and a second voltage at a second pair of measurement electrodes; determining the value indicative of the impedance of the suspension by correlating the first impedance measurement and the second impedance measurement.

### CROSS-REFERENCE TO RELATED APPLICATIONS

This application is a national phase entry under 35 U.S.C. § 371 of International Application PCT/EP2019/083114, filed Nov. 29, 2019, which claims priority to DE Patent Application No. 10 2018 130 487.0, filed Nov. 30, 2018, all of which are herein incorporated by reference in their entirety.

### TECHNICAL FIELD

The present invention pertains to the field of impedance spectroscopy of suspensions, in particular cell suspensions. In particular, the present invention relates to determining the impedance of a suspension or determining a value indicative of the impedance of a suspension. Still more particularly, the present invention relates to a method and a sensor for determining a value indicative of the impedance of a suspension.

### BACKGROUND

Electrical impedance spectroscopy methods are used as measuring methods for the non-destructive in-situ and in-vivo determination of frequency-dependent passive electrical properties of suspensions. The term suspension refers to the distribution of small particles of a substance or mixture of substances in a liquid. An example of a suspension analyzed by electrical impedance spectroscopy methods is a substance consisting of a liquid and biological cells contained therein, collectively referred to herein as cell population. The above-mentioned frequency-dependent passive electrical properties of the cell population can provide information, among other things, about the number of living cells and/or the size of the cells and/or the homogeneity of the cells. Previous sensors and impedance spectroscopy methods are not always completely satisfactory with regard to the accuracy of the measurement results. Also, the quality of the measurement results can vary greatly over a wide frequency range.

Accordingly, it would be desirable to provide a method and sensor for determining a value indicative of the impedance of a suspension that has high measurement accuracy and permits reliable measurements over a wide range of frequencies.

### SUMMARY

Exemplary embodiments of the invention comprise a method for determining a value indicative of the impedance of a suspension in the framework of an impedance spectroscopy, comprising the following steps: generating an excitation current through the suspension, the excitation current oscillating at an excitation frequency; determining a first impedance measurement value on the basis of the excitation current and a first voltage at a first pair of measurement electrodes; determining a second impedance measurement value on the basis of the excitation current and a second voltage at a second pair of measurement electrodes; determining the value indicative of the impedance of the suspension by correlating the first impedance measurement value and the second impedance measurement value.

Exemplary embodiments of the invention allow determining the value indicative of the impedance of the suspension with high measurement accuracy. By determining a first impedance measurement value and a second impedance measurement value and by correlating the first impedance measurement value and the second impedance measurement value, it is possible to reduce the susceptibility of the measurement result to interfering influences or interferences. In particular, it is possible to eliminate interferences, which affect the first impedance measurement value and the second impedance measurement value in the same or a similar way, to a large extent or completely from the measurement result by correlating the first and second impedance measurement values. For example, by a differential consideration of the first impedance measurement value and the second impedance measurement value, low-order interferences can be largely or completely eliminated from the measurement result. It is possible to significantly reduce the influence of parasitic capacitances and/or other parasitic interfering influences on the measurement result. The measurement accuracy of individual measurements and/or the bandwidth of the frequency range of usable measurements can be increased.

More accurate and/or more reliable values, indicating the impedance of a suspension, can be determined.

The method comprises the steps of determining a first impedance measurement value on the basis of the excitation current and a first voltage at a first pair of measurement electrodes and determining a second impedance measurement value on the basis of the excitation current and a second voltage at a second pair of measurement electrodes. The first and second pairs of measurement electrodes are different pairs, i.e. the first and second pairs of measurement electrodes differ in at least one measurement electrode. This in turn means that the sensor used has at least three measurement electrodes. The first voltage and the second voltage are thus measurement values for different cell geometries, i.e. measurement values for different measurement cells in the suspension. The term measurement cell refers to the entirety of all influences of the suspension on the arrangement of two specific measurement electrodes. In particular, the terms first voltage and second voltage refer to a first measured voltage and a second measured voltage. Determining the first impedance measurement value may comprise a first voltage measurement, and determining the second impedance measurement value may comprise a second voltage measurement.

The term suspension describes a distribution of particles in a liquid. In particular, the term suspension denotes a suspension containing particles with an impedance >0. The particles may be non-living particles, such as carbon particles. However, they can also be living or partially living particles, such as cells. The suspension may be a cell population. The term cell population is used herein for a collection of biological cells in a carrier liquid. In particular, the term cell population is used for collections of biological cells that have a significant proportion of living cells. By determining one or more values indicating the impedance of the suspension, one or more properties of the suspension can be derived.

According to a further embodiment, the first pair of measurement electrodes comprises a first measurement electrode and a second measurement electrode, and the second pair of measurement electrodes comprises the first measurement electrode and a third measurement electrode. In other words, the first pair of measurement electrodes and the second pair of measurement electrodes consist of a total of three measurement electrodes. In this way, two pairs of measurement electrodes can be provided with a minimum total number of measurement electrodes. As a result, the method can be carried out using a sensor with few components.

According to a further embodiment, the first pair of measurement electrodes comprises a first measurement electrode and a second measurement electrode, and the second pair of measurement electrodes comprises a third measurement electrode and a fourth measurement electrode. In this way, the first pair of measurement electrodes and the second pair of measurement electrodes are independent of each other. The measurements on the first pair of measurement electrodes and the second pair of measurement electrodes can be carried out separately. They can also be carried out simultaneously, depending on the downstream signal processing. Separating the two pairs of measurement electrodes can simplify downstream signal processing. In addition, the geometry of the measurement cell of the first pair of measurement electrodes and the geometry of the measurement cell of the second pair of measurement electrodes can have a higher degree of independence than in the previously mentioned case of a total of three measurement electrodes. In this way it may be possible to remove interferences from the measurement results even better.

According to a further embodiment, determining the value indicative of the impedance of the suspension comprises determining the difference between the first impedance measurement value and the second impedance measurement value. Alternatively, determining the value indicative of the impedance of the suspension may comprise determining the difference between a first adjusted impedance value and a second adjusted impedance value, wherein the first adjusted impedance value and the second adjusted impedance value are obtained by applying a correction function to the first impedance measurement value and the second impedance measurement value. Determining the difference is a low-complexity but effective way to remove a significant portion of the interfering influences on the measurement values. Thus, with relatively little computational expenditure, a great improvement of the measurement accuracy of the value indicative of the impedance of a suspension can be achieved. The above-mentioned correction function can represent the transmission behavior of the measurement arrangement. In this way, the influence of the measurement arrangement on the measured signals, such as additional signal propagation times, amplifications, losses, etc., can be taken into account.

According to a further embodiment, determining the value indicative of the impedance of the suspension comprises determining the difference between a first geometry factor and a second geometry factor, wherein the first geometry factor represents the measurement geometry of the first pair of measurement electrodes and wherein the second geometry factor represents the measurement geometry of the second pair of measurement electrodes. By the afore-mentioned determining of the difference, the different geometric arrangements of the two pairs of measurement electrodes can be taken into account in a low-complexity, but effective way. The fact that the first impedance measurement value and the second impedance measurement value are based on different measurement cells due to the use of different pairs of measurement electrodes can thus be taken into account. The first geometry factor and the second geometry factor may be calculated prior to determining the value indicative of the impedance of the suspension. It is also possible that the first geometry factor and the second geometry factor are determined experimentally in a calibration phase prior to the actual implementation of the method. The first geometry factor and the second geometry factor can be assumed to be constant for determining multiple values indicative of the impedance of the suspension in the framework of an impedance spectroscopy.

According to a further embodiment, determining the value indicative of the impedance of the suspension is carried out according to the following formula:

$${Z = k \frac{1}{( {λ}_{1} - {λ}_{2 } )} {( {G}_{el}^{- 1} ( {Z}_{sig} ) \vert}_{1} - {G}_{el}^{- 1} ( {Z}_{sig} ) \vert}_{2} ) ,$$

wherein Z_{sig}|₁ denotes the first impedance measurement value, Z_{sig}|₂ denotes the second impedance measurement value, Gₑₗ⁻¹ denotes a correction function representing the transmission behavior of the measurement arrangement, λ₁ denotes a first geometry factor representing the measurement geometry of the first pair of measurement electrodes, λ2 denotes a second geometry factor representing the measurement geometry of the second pair of measurement electrodes, and k denotes a proportionality constant. The first geometry factor λ₁ and the second geometry factor λ₂ can represent the geometry of the different measurement cells described above. The correction function Gₑₗ⁻¹ is referred to as inverse function, io which is indicated by the superscript −1. This notation takes into account the fact that Z_{sig}|₁ and Z_{sig}|₂ are the impedance measurement values after passing through the measurement arrangement. The transmission behavior of the measurement arrangement can be described by a function Gₑₗ. Thus, the correction function Gₑₗ⁻¹ allows back-calculation as to which impedance measurement values were applied directly to the measurement electrodes. The values to be used for k, Gₑₗ⁻¹, λ₁ and λ₂ can be calculated or determined in a calibration phase or partially calculated and partially determined in a calibration phase.

According to a further embodiment, the method comprises: measuring the first voltage at the first pair of measurement electrodes, and measuring the second voltage at the second pair of measurement electrodes, wherein said measuring of the first voltage and said measuring of the second voltage are performed substantially simultaneously. In this way, it is possible to keep small or completely eliminate the influence of the time variability of the interfering influences on the determination of the value indicative of the impedance of a suspension.

According to an alternative embodiment, the method comprises: measuring the first voltage on the first pair of measurement electrodes, and measuring the second voltage on the second pair of measurement electrodes, wherein said measuring of the first voltage and said measuring of the second voltage are performed in a time-shifted manner. In particular, the voltage measured at the first pair of measurement electrodes and the voltage measured at the second pair of measurement electrodes can be provided sequentially to the downstream signal processing. For this purpose, for example, appropriate switches can be provided between the first pair of measurement electrodes and the second pair of measurement electrodes and the downstream signal processing. In this way, the downstream signal processing can be implemented with comparatively few components and can be made compact and energy-efficient.

Irrespective of whether the measurement of the first voltage and the measurement of the second voltage are substantially simultaneous or time-shifted, the measurement of the first voltage and the measurement of the second voltage each occur when the excitation current through the suspension, oscillating at the excitation frequency, is generated. In other words, a voltage is measured at the measurement electrodes while the excitation current, oscillating with the excitation frequency, is applied to the suspension. Accordingly, the electrical response of the suspension is measured when the excitation current, oscillating at the excitation frequency, is applied. The excitation current can also be measured. It is also possible that the excitation current is known or assumed to be known as a result of a known generation mechanism.

According to a further embodiment, the excitation frequency of the excitation current is between 50 kHz and 20 MHz. With an excitation current in this frequency range, particularly relevant values indicative of the impedance of a cell population can be determined, which in particular permit a good conclusion as to the quantities and/or the size and/or the homogeneity of the living cells of the cell population. The frequency range mentioned lies in the so-called β-dispersion region of many cell populations, which will be discussed in more detail below.

According to a further embodiment, determining the first impedance measurement value and determining the second impedance measurement value comprises: sampling the excitation current, sampling the first voltage and sampling the second voltage. Sampling the excitation current, the first voltage and the second voltage helps to determine the value indicative of the impedance of a suspension with high accuracy over a wide frequency range. Sampling the excitation current, sampling the first voltage and sampling the second voltage allow sampling values to be generated at precisely determined times. These time-discretized sampling values can be analyzed and correlated to each other after sampling, without the need for the signal processing following the sampling to be real-time capable. A comparatively large database, clearly defined in the time dimension by the sampling, can be used to determine the value indicative of the impedance of the suspension with high accuracy. Compared to earlier approaches, which are based on determining characteristic properties of a suspension by means of complicated analog signal processing, sampling allows minimizing the interferences after sampling, since the signal processing of the discretized sampling values can be designed very robustly. The interferences between the measurement of excitation current, first voltage and second voltage and the sampling of excitation current, first voltage and second voltage can be kept very low. Furthermore, the sampling of the excitation current, the first voltage and the second voltage can be adapted to the excitation frequency, which enables high accuracy of the sampling at the relevant frequencies and a spectral limitation of the interferences.

The sampling of the excitation current, the sampling of the first voltage, and the sampling of the second voltage may be a sampling of values derived from excitation current, first voltage, and second voltage. For example, a first signal can be generated for the excitation current that represents the excitation current. This first signal can be a voltage signal, for example. The first signal can then be sampled directly or after amplification. Such signal processing also falls under the term of sampling the excitation current in the sense of the present document. It is further possible that the first voltage between the first pair of measurement electrodes is sampled in the form of a second signal. This second signal may also be sampled either directly or amplified. As with the sampling of the excitation current, such preprocessing of the second signal also falls under the term sampling of the first voltage. It is further possible that the second voltage is sampled between the second pair of measurement electrodes in the form of a third signal. This third signal may also be sampled either directly or amplified. As in the case of sampling the excitation current, such preprocessing of the third signal also falls under the term of sampling the second voltage.

According to a further embodiment, the method further comprises the following steps: setting a first sampling rate for sampling the excitation current, setting a second sampling rate for sampling the first voltage, and setting a third sampling rate for sampling the second voltage. In particular, setting the first sampling rate and/or setting the second sampling rate and/or setting the third sampling rate can be based on the excitation frequency of the excitation current. In this case, the first sampling rate, the second sampling rate and the third sampling rate may be the same or different. The setting of the first sampling rate, the setting of the second sampling rate and the setting of the third sampling rate allow the determination of the value indicative of the impedance of the suspension to be adapted to the general conditions of a current measuring operation, in particular to the excitation frequency of the excitation current for the current measuring operation. In this way, it is possible to use optimized sampling rates for each measurement operation, in particular to set a sampling rate that is optimized in terms of accuracy and/or signal processing complexity. The first sampling rate, the second sampling rate and the third sampling rate are used for sampling the excitation current, for sampling the first voltage and for sampling the second voltage. Accordingly, the first sampling rate, the second sampling rate, and the third sampling rate are set before sampling the excitation current, the first voltage and the second voltage. The wording of setting the first sampling rate and setting the second sampling rate and setting the third sampling rate also includes setting one sampling rate and using that one sampling rate as first sampling rate, as second sampling rate and as third sampling rate.

According to a further embodiment, the first sampling rate, the second sampling rate and the third sampling rate are set to at least 4 times the excitation frequency of the excitation current, in particular to substantially 4 times the excitation frequency of the excitation current. Using at least 4 times the excitation frequency of the excitation current for sampling the excitation current, first voltage, and second voltage ensures that the excitation current, first voltage, and second voltage are sampled very accurately and that no signal information is lost around the excitation frequency. The sampling theorem is over-satisfied by a reassuring margin. In particular, the first sampling rate, the second sampling rate and the third sampling rate can be set to substantially 4 times the excitation frequency of the excitation current or even exactly 4 times the excitation frequency of the excitation current.

According to a further embodiment, the step of determining the first impedance measurement value comprises performing a first complex Fourier transform on the basis of the sampling values of the excitation current and the sampling values of the first voltage, and the step of determining the second impedance measurement value comprises performing a second complex Fourier transform on the basis of the sampling values of the excitation current and the sampling values of the second voltage. In particular, a complex discrete Fourier transform may be used. The sampling values of the excitation current, the sampling values of the first voltage and the sampling values of the second voltage can be regarded as respective real parts of a complex current or voltage signal. By means of a complex Fourier transform, which considers sampled current and voltage values together, the complex impedance between excitation current and first voltage or between excitation current and second voltage can be determined. In particular, complex impedances at the excitation frequency can be determined, which can form the basis for the first impedance measurement value or the second impedance measurement value.

According to a further embodiment, the method further comprises: determining a third impedance measurement value on the basis of the excitation current and a third voltage at a third pair of measurement electrodes, determining the value indicative of the impedance of the suspension by correlating the first impedance measurement value, the second impedance measurement value and the third impedance measurement value. By determining a third impedance measurement value and correlating the first, second, and third impedance measurement values, interferences can be removed from the measurement results to an even greater degree. In particular, by using three impedance measurement values from three different pairs of measurement electrodes, the interferences of a higher order can be reduced or even eliminated than is possible with the use of two different pairs of measurement electrodes. The three pairs of measurement electrodes form three different geometric arrangements in the suspension. Thus, measurement values are available for three measurement cells. The three pairs of measurement electrodes can be formed by a total of four or more measurement electrodes. In particular, it is possible that a total of four, five or six measurement electrodes are used, from which three different pairs of measurement electrodes are used for the respective determination of an impedance measurement value.

According to a further embodiment, determining the value indicative of the impedance of the suspension comprises determining a first difference between the first impedance measurement value and the second impedance measurement value and determining a second difference between the first impedance measurement value and the third impedance measurement value and determining a third difference between the second impedance measurement value and the third impedance measurement value. Analogous to the case of two pairs of measurement electrodes described above, determining said first, second and third differences represent low-complexity, but effective measures to remove a significant portion of the interfering influences on the measurement values.

According to an alternative embodiment, determining the value indicative of the impedance of the suspension comprises determining a first difference between a first adjusted impedance value and a second adjusted impedance value and determining a second difference between the first adjusted impedance value and a third adjusted impedance value, and determining a third difference between the second adjusted impedance value and the third adjusted impedance value, wherein the first adjusted impedance value, the second adjusted impedance value and the third adjusted impedance value are obtained by applying a correction function to the first impedance measurement value, the second impedance measurement value and the third impedance measurement value. According to a further embodiment, the correction function can represent the transmission behavior of the measurement arrangement.

According to a further embodiment, determining the value indicative of the impedance of the suspension comprises determining a first difference between a first geometry factor and a second geometry factor and determining a second difference between the first geometry factor and a third geometry factor and determining a third difference between the second geometry factor and the third geometry factor, wherein the first geometry factor represents the measurement geometry of the first pair of measurement electrodes, the second geometry factor represents the measurement geometry of the second pair of measurement electrodes, and the third geometry factor represents the measurement geometry of the third pair of measurement electrodes. By means of the above-mentioned determined differences, the different geometric arrangements of the three pairs of measurement electrodes can be taken into account in a low-complexity, but effective way.

According to a further embodiment, determining the value indicative of the impedance of the suspension is carried out according to the following formula:

$${Z}^{2 } = {k}_{2} \frac{{{{λ}_{3} ( {G}_{el}^{- 1} ( {Z}_{sig} ) \vert}_{2} - {G}_{el}^{- 1} ( {Z}_{sig} ) \vert}_{1} )}{( {λ}_{1} - {λ}_{2} ) ( {λ}_{1} - {λ}_{3} ) ( {λ}_{2} - {λ}_{3} )} ++ {k}_{2} \frac{{{{λ}_{2} ( {G}_{el}^{- 1} ( {Z}_{sig} ) \vert}_{1} - {G}_{el}^{- 1} ( {Z}_{sig} ) \vert}_{3} )}{( {λ}_{1} - {λ}_{2} ) ( {λ}_{1} - {λ}_{3} ) ( {λ}_{2} - {λ}_{3} )} ++ {k}_{2} \frac{{{{λ}_{1} ( {G}_{el}^{- 1} ( {Z}_{sig} ) \vert}_{3} - {G}_{el}^{- 1} ( {Z}_{sig} ) \vert}_{2} )}{( {λ}_{1} - {λ}_{2} ) ( {λ}_{1} - {λ}_{3} ) ( {λ}_{2} - {λ}_{3} )} ,$$

wherein Z_{sig}|₁ denotes the first impedance measurement value, Z_{sig}|₂ denotes the second impedance measurement value, Z_{sig}|₃ denotes the third impedance measurement value, Gₑₗ⁻¹ denotes a correction function that represents the transmission behavior of the measurement arrangement, λ₁ denotes a first geometry factor that represents the measurement geometry of the first pair of measurement electrodes, λ₂ denotes a second geometry factor that represents the measurement geometry of the second pair of measurement electrodes, λ₃ denotes a third geometry factor that represents the measurement geometry of the third pair of measurement electrodes, and k₂ denotes a proportionality constant. The values to be used for k₂, Gₑₗ⁻¹, λ₁, λ₂, λ₃ may be calculated or determined in a calibration phase or may be partially calculated and partially determined in a calibration phase.

According to a further embodiment, determining the value indicative of the impedance of the suspension is performed according to the following formula:

$$Z = {k}_{2} \frac{{{{λ}_{3} ( {G}_{el}^{- 1} ( {Z}_{sig} ) \vert}_{2} - {G}_{el}^{- 1} ( {Z}_{sig} ) \vert}_{1} )}{( {λ}_{1} - {λ}_{2} ) ( {λ}_{1} - {λ}_{3} ) ( {λ}_{2} - {λ}_{3} )} ++ {k}_{2} \frac{{{{λ}_{2} ( {G}_{el}^{- 1} ( {Z}_{sig} ) \vert}_{1} - {G}_{el}^{- 1} ( {Z}_{sig} ) \vert}_{3} )}{( {λ}_{1} - {λ}_{2} ) ( {λ}_{1} - {λ}_{3} ) ( {λ}_{2} - {λ}_{3} )} ++ {k}_{2} \frac{{{{λ}_{1} ( {G}_{el}^{- 1} ( {Z}_{sig} ) \vert}_{3} - {G}_{el}^{- 1} ( {Z}_{sig} ) \vert}_{2} )}{( {λ}_{1} - {λ}_{2} ) ( {λ}_{1} - {λ}_{3} ) ( {λ}_{2} - {λ}_{3} )} ,$$

wherein Z_{sig}|₁ denotes the first impedance measurement value, Z_{sig}51 ₂ denotes the second impedance measurement value, Z_{sig}|₃ denotes the third impedance measurement value, Gₑₗ⁻¹ denotes a correction function that represents the transmission behavior of the measurement arrangement, λ₁ denotes a first geometry factor that represents the measurement geometry of the first pair of measurement electrodes, λ₂ denotes a second geometry factor that represents the measurement geometry of the second pair of measurement electrodes, λ₃ denotes a third geometry factor that represents the measurement geometry of the third pair of measurement electrodes, and k₂ denotes a proportionality constant. The values to be used for k₂, Gₑₗ⁻¹, λ₁, λ₂, and λ₃ may be calculated or determined in a calibration phase, or may be partially calculated and partially determined in a calibration phase.

Exemplary embodiments of the invention further comprise a method for determining a value indicative of the impedance of a suspension in the framework of an impedance spectroscopy, comprising the steps of: generating an excitation voltage, oscillating at an excitation frequency, applied to the suspension; determining a first impedance measurement value on the basis of the excitation voltage and a first current through a first pair of measurement electrodes; determining a second impedance measurement value on the basis of the excitation voltage and a second current through a second pair of measurement electrodes; determining the value indicative of the impedance of the suspension by correlating the first impedance measurement value and the second impedance measurement value. Generating an excitation voltage and determining first and second impedance measurement values on the basis of the excitation voltage and the first and second currents constitutes an alternative embodiment to generating an excitation current and determining first and second impedance measurement values on the basis of the excitation current and the first and second voltages, as described above. In other words, alternative embodiments of the invention involve applying an excitation voltage to the suspension and using current flows through measurement electrodes to determine the impedance measurement values. For example, it is possible to apply the excitation voltage such that no current flow passes through a pair of excitation electrodes. In other words, the pair of excitation electrodes can be used only to provide for a time-varying potential difference in the suspension. Furthermore, the first pair of measurement electrodes and the second pair of measurement electrodes may each be connected to each other, for example, via a measuring resistor or via a measuring capacitor. Thus, a closed AC circuit is present through the measuring resistor/measuring capacitor and the suspension, respectively, and the voltage at the measuring resistor/measuring capacitor can be tapped as a measure of the current flowing through the respective pair of measurement electrodes. The additional features, modifications and technical effects described above with reference to the method using an excitation current apply analogously to the method using an excitation voltage and are hereby explicitly disclosed for this alternative solution.

Exemplary embodiments of the invention further comprise a method for deriving at least one characteristic property of a suspension, comprising the following steps: performing the method for determining a value indicative of the impedance of a suspension in accordance with any of the embodiments described above a plurality of times, wherein, for the plurality of times the method is performed, a plurality of different excitation frequencies are made use of and a plurality of values indicative of the impedance of the suspension are determined for the plurality of different excitation frequencies; deriving a plurality of values indicative of the permittivity of the suspension on the basis of the plurality of values indicative of the impedance of the suspension; and deriving the at least one characteristic property of the suspension by correlating the plurality of values indicative of the permittivity of the suspension. Said correlating may include forming a difference between two values indicative of the permittivity of the suspension, and/or determining the slope of a curve drawn through the plurality of values indicative of the permittivity of the suspension, and/or determining an inflection point of a curve drawn through the plurality of values indicative of the permittivity of the suspension, and/or determining further characteristic properties of the plurality of values indicative of the permittivity of the suspension. By means of these characteristic properties of the plurality of values obtained, conclusions can be drawn about characteristic properties of the suspension, in particular about characteristic properties of a cell population. By determining the plurality of values indicating the permittivity of the suspension with high accuracy over a comparatively large frequency range, the characteristic properties of the suspension can also be determined with high accuracy. The plurality of values indicating the permittivity of the suspension may be capacitance values or permittivity values.

According to a further embodiment, said method for determining a value indicative of the impedance of a suspension is performed for between 2 and 50 different excitation frequencies. In particular, said method for determining a value indicative of the impedance of a suspension can be performed for between 10 and 40 different excitation frequencies, further in particular for between 20 and 30 different excitation frequencies. It has been found that for the above-mentioned number of runs of the method and the corresponding number of values indicative of the impedance of the suspension, in particular for between 10 and 40 different excitation frequencies and further in particular for between 20 and 30 different excitation frequencies, a good compromise can be achieved between the complexity of the method for deriving at least one characteristic property of the suspension and the accuracy of the results with respect to the at least one characteristic property of the suspension. In particular, for between 10 and 40 different excitation frequencies, or further in particular for between 20 and 30 different excitation frequencies, particularly meaningful curves of the values indicative of the permittivity of the suspension, in particular of the permittivity itself, can be generated. The afore-mentioned number of values allows the generation of curves versus the excitation frequency with sufficient accuracy in order to determine characteristic properties such as slope and inflection point with high accuracy. These embodiments apply in particular to deriving at least one characteristic property of a cell population.

According to a further embodiment, the different excitation frequencies are from a frequency range from 100 kHz to 10 MHz. The expression that the different excitation frequencies are from said frequency range means that at least said frequency range is covered by the different excitation frequencies. This in turn means that the lowest excitation frequency is 100 kHz or less and that the highest excitation frequency is 10 MHz or more. In other words, the lowest excitation frequency and the highest excitation frequency form an intermediate excitation frequency span that includes at least the range between 100 kHz to 10 MHz. With the values indicative of the impedance of the suspension for the excitation frequencies of 100 kHz to 10 MHz, one or more characteristic properties of a large number of suspensions, in particular of a large number of cell populations, can be determined with high accuracy.

In a further embodiment, the different excitation frequencies are from a frequency range from 50 kHz to 20 MHz. This allows the derivation of the at least one characteristic property of the suspension to be further refined. This applies in particular to the derivation of the at least one characteristic property of a cell population. Exemplary embodiments of the above-described method for determining a value indicative of the impedance of a suspension allow, due to the increased measurement accuracy and/or the increased reliability of the measurement results, an extension of the frequency range of the impedance spectroscopy and, thus, a more comprehensive determination of one or more characteristic properties of the suspension, without having to resort to additional and more complex methods. It is also possible that the method can be applied to an extended range of suspensions, in particular to an extended range of cell populations.

According to a further embodiment, deriving the at least one characteristic property of the suspension includes generating a curve of the values indicative of the permittivity of the suspension over the different excitation frequencies. In other words, a curve can be fitted through the values indicating the permittivity of the suspension versus the excitation frequency. In doing so, a so-called Cole-Cole fitting can be applied. From the resulting curve or from the resulting course, it is then possible to determine characteristics such as differences between end values, slopes and inflection points. The values indicating the permittivity of the suspension can directly be the determined values or can be calibrated versions of the determined values.

According to a further embodiment, the suspension is a cell population. In a further embodiment, the at least one characteristic property of the cell population comprises at least one property of number of the living cells, size of the cells, and homogeneity of the cells.

Exemplary embodiments of the invention further comprise a sensor for determining a value indicative of the impedance of a suspension, comprising: an oscillator circuit; a pair of excitation electrodes coupled to the oscillator circuit, wherein an excitation current through the suspension, oscillating at an excitation frequency, can be generated across the pair of excitation electrodes by means of the oscillator circuit; at least three measurement electrodes for measuring a first voltage in the suspension between a first pair of the at least three measurement electrodes and a second voltage in the suspension between a second pair of the at least three measurement electrodes; and a data processing device configured to determine a first impedance measurement value on the basis of the excitation current and the first voltage, to determine a second impedance measurement value on the basis of the excitation current and the second voltage, and to determine the value indicative of the impedance of the suspension by correlating the first impedance measurement value and the second impedance measurement value. The additional features, modifications and technical effects described above with respect to the method for determining a value indicative of the impedance of a suspension apply analogously to the sensor for determining a value indicative of the impedance of a suspension.

According to a further embodiment, the at least three measurement electrodes are arranged between the pair of excitation electrodes. In this way, the excitation current is applied in high strength along the measurement electrodes, and the ratio of useful signal to interfering influences is high compared to other geometric arrangements. Moreover, such an arrangement enables a compact design of the sensor.

According to another embodiment, the first pair of the at least three measurement electrodes comprises a first measurement electrode and a second measurement electrode, and the second pair of the at least three measurement electrodes comprises the first measurement electrode and a third measurement electrode.

According to an alternative embodiment, the at least three measurement electrodes are at least four measurement electrodes, wherein the first pair of the at least four measurement electrodes comprises a first measurement electrode and a second measurement electrode, and wherein the second pair of the at least four measurement electrodes comprises a third measurement electrode and a fourth measurement electrode.

The statements given above with respect to the different numbers of electrodes in the context of the method for determining a value indicative of the impedance of a suspension apply analogously to the sensor for determining a value indicative of the impedance of a suspension.

According to a further embodiment, the third and fourth measurement electrodes are arranged between the first and second measurement electrodes. Such an arrangement allows a high overlap of the measurement cells. As a result, there is a high probability that interfering influences will affect the two measurement cells in a very similar or identical manner. This, in turn, allows for an optimized removal of the interfering influences from the measurement values by said correlating of the first impedance measurement value and the second impedance measurement value, as discussed in detail above. Furthermore, such an arrangement allows for a compact design of the sensor.

According to a further embodiment, the third and fourth measurement electrodes are arranged on a different side of the sensor than the first and second measurement electrodes. In this way, the mutual influence of the measurement electrodes can be kept small. A largely independent determination of the first impedance measurement value and the second impedance measurement value is thus rendered possible. Depending on the general conditions, i.e. depending on the suspension to be investigated and the existing interfering influences, particularly good measurement results can be achieved in this way in individual cases.

According to a further embodiment, the data processing device is configured to determine the value indicative of the impedance of the suspension via determining the difference between the first impedance measurement value and the second impedance measurement value.

According to an alternative embodiment, the data processing device is configured to determine the value indicative of the impedance of the suspension via determining the difference between a first adjusted impedance value and a second adjusted impedance value, wherein the data processing device is configured to obtain the first adjusted impedance value and the second adjusted impedance value by applying a correction function to the first impedance measurement value and the second impedance measurement value. In a further embodiment, the correction function represents the transmission behavior of the measurement arrangement.

According to a further embodiment, the data processing device is configured to determine the value indicative of the impedance of the suspension via determining the difference between a first geometry factor and a second geometry factor, wherein the first geometry factor represents the measurement geometry of the first pair of the at least three measurement electrodes and wherein the second geometry factor represents the measurement geometry of the second pair of the at least three measurement electrodes.

According to a further embodiment, the data processing device is configured to determine the value indicative of the impedance of the suspension according to the following formula:

$${Z = k \frac{1}{( {λ}_{1} - {λ}_{2 } )} {( {G}_{el}^{- 1} ( {Z}_{sig} ) \vert}_{1} - {G}_{el}^{- 1} ( {Z}_{sig} ) \vert}_{2} ) ,$$

wherein Z_{sig}|₁ denotes the first impedance measurement value, Z_{sig}|₂ denotes the second impedance measurement value, Gₑₗ⁻¹ denotes a correction function representing the transmission behavior of the measurement arrangement, λ₁ denotes a first geometry factor representing the measurement geometry of the first pair of the at least three measurement electrodes, λ₂ denotes a second geometry factor representing the measurement geometry of the second pair of the at least three measurement electrodes, and k denotes a proportionality constant.

According to a further embodiment, the oscillator circuit is arranged to set the excitation frequency in a frequency range from 100 kHz to 10 MHz. That is, the oscillator circuit is capable of setting the excitation frequency in a frequency range from 100 kHz to 10 MHz. This expression does not exclude that the oscillator circuit is capable to set the excitation frequency beyond the specified frequency range. Rather, the expression means that the oscillator circuit is capable to set the excitation frequency at least in the frequency range from 100 kHz to 10 MHz. In particular, the oscillator circuit may be arranged to set the excitation frequency at least in a frequency range from 50 kHz to 20 MHz.

According to a further embodiment, the sensor further comprises: a first sampling circuit coupled to at least one of the pair of excitation electrodes and operative to provide sampling values of the excitation current; and at least one further sampling circuit coupled to the at least three measurement electrodes and providing, in operation, sampling values for the first voltage and the second voltage; wherein the data processing device is coupled to the first sampling circuit and the at least one further sampling circuit. For obtaining data for the first impedance measurement value or for the second impedance measurement value, the first sampling circuit and the respectively required further sampling circuit operate simultaneously, i.e. they generate the sampling values for the excitation current and the sampling values for the first voltage/the second voltage in the same period of time. Further, the first sampling circuit and the respectively required further sampling circuit operate when the excitation current is applied, i.e., the first sampling circuit and the required further sampling circuit operate in the same period of time as the oscillator circuit.

The term coupled is used herein to indicate that a signal or electrical parameter can be transmitted from one entity to another, i.e., that some type of connection exists between the entities. However, other components, such as amplifiers, transformers, or other electrical components, may be interposed.

The first sampling circuit may be coupled to either the first excitation electrode or the second excitation electrode. It may also be coupled to both excitation electrodes. In general, the first sampling circuit may be coupled to one or both excitation electrodes in any suitable manner such that measurement of the excitation current is possible.

According to a further embodiment, the first sampling circuit and the at least one further sampling circuit are synchronized with respect to their sampling times.

According to a further embodiment, the first sampling circuit and the at least one further sampling circuit are coupled to the data processing device via a data storage, wherein the data storage in particular has a data recording rate of at least 1 Gbit/s. The data recording rate of at least 1 Gbit/s describes a possible data recording rate of at least 1 Gbit/s. The data do not have to be recorded by the data storage at this rate. Since the sampling frequency may depend on the excitation frequency, different data recording rates at the data storage may result in operation for different excitation frequencies.

According to a further embodiment, the data processing device is configured to determine the first impedance measurement value by means of a first complex

Fourier transform on the basis of the sampling values of the excitation current and the sampling values of the first voltage, and to determine the second impedance measurement value by means of a second complex Fourier transform on the basis of the sampling values of the excitation current and the sampling values of the second voltage.

According to a further embodiment, the first sampling circuit is coupled to the at least one of the pair of excitation electrodes via a first amplifier circuit, and the at least one further sampling circuit is coupled to the at least three measurement electrodes via at least one further amplifier circuit.

According to a further embodiment, a measuring element, in particular a measuring resistor, is coupled to the at least one of the pair of excitation electrodes, and the first sampling circuit is configured to provide the sampling values for the excitation current on the basis of the voltage drop across the measuring element. For reasons of symmetry, both excitation electrodes may each be coupled to a measuring element, in particular to a measuring resistor, even if the first sampling circuit is coupled to only one of the measuring elements.

According to a further embodiment, the oscillator circuit is coupled to the pair of excitation electrodes via a transformer. Thus, galvanic decoupling between the oscillator circuit and the excitation electrodes can be achieved.

According to a further embodiment, the transformer has a parallel capacitance of 0.5 pF to 10 pF, in particular a parallel capacitance of 1 pF to 5 pF. The parallel capacitance may be a discrete component, such as a capacitor arranged parallel to the transformer. However, it is also possible that the parallel capacitance is a parasitic capacitance of the transformer, wherein the transformer is designed such that the parallel capacitance is in the specified range of values. The term parallel capacitance refers to a coupling capacitance between the primary side and the secondary side of the transformer. With a parallel capacitance, the influence of interfering coupling capacitances, such as may be present between the electrodes and the wall of a container for the suspension or between the electrodes and other sensors present, can be kept low. Since, in the presence of the capacitance parallel to the transformer and other coupling capacitances, the smaller capacitance is often determinative, the influence of an undesirable interfering coupling capacitance can be reduced to the magnitude of the parallel capacitance.

According to a further embodiment, the sensor further comprises a control unit coupled to the oscillator circuit and causing the oscillator circuit in operation to successively generate excitation currents through the suspension that oscillate at different excitation frequencies. Thus, the control unit can initiate different measurement runs by means of which impedance spectroscopy can be performed for the suspension. Furthermore, the control unit may be coupled to the first sampling circuit and the at least one further sampling circuit and be configured to transmit the currently generated excitation frequency to the first and the at least one further sampling circuit. The first sampling circuit and the at least one further sampling circuit may accordingly adjust the sampling rate. The control unit may also be arranged to transmit the currently generated excitation frequency to the data processing device.

Exemplary embodiments of the invention further comprise a sensor for determining a value indicative of the impedance of a suspension, comprising: an oscillator circuit; a pair of excitation electrodes coupled to the oscillator circuit, wherein an excitation voltage, oscillating at an excitation frequency, applied to the suspension can be generated across the pair of excitation electrodes by means of the oscillator circuit; at least three measurement electrodes for measuring a first current in the suspension between a first pair of the at least three measurement electrodes and a second current in the suspension between a second pair of the at least three measurement electrodes; and a data processing device configured to determine a first impedance measurement value on the basis of the excitation voltage and the first current, to determine a second impedance measurement value on the basis of the excitation voltage and the second current, and to determine the value indicative of the impedance of the suspension by correlating the first impedance measurement value and the second impedance measurement value. The additional features, modifications and technical effects described above with respect to the sensor, whose excitation electrodes an excitation current can be generated with, apply analogously to the sensor, whose excitation electrodes an excitation voltage can be generated with, and are hereby explicitly disclosed for this alternative solution. Furthermore, the above considerations for the method using an excitation voltage are analogously applicable to the sensor, whose excitation electrodes an excitation voltage can be generated with.

Exemplary embodiments of the invention further comprise a computer program or computer program product comprising program instructions which, when executed on a data processing system, perform a method according to any of the embodiments described above. In this regard, the individual steps of the method may be initiated by the program instructions and executed by other components or may be executed in the data processing system itself.

### BRIEF DESCRIPTION OF THE DRAWINGS

Further exemplary embodiments of the invention are described below with reference to the accompanying drawings.

FIG. 1 shows a sensor for determining a value indicative of the impedance of a suspension according to an exemplary embodiment of the invention in a side view;

FIG. 2 shows a sensor according to an exemplary embodiment of the invention, shown in part as a block diagram and in part as a circuit diagram;

FIG. 3 shows a sensor, modified as compared to FIG. 2, in accordance with a further exemplary embodiment of the invention, shown in part as a block diagram and in part as a circuit diagram;

FIG. 4 shows a sensor for determining a value indicative of the impedance of a suspension according to a further exemplary embodiment of the invention in a perspective view;

FIG. 5 shows a sensor for determining a value indicative of the impedance of a suspension according to a further exemplary embodiment of the invention in a side view;

FIG. 6 shows a sensor according to an exemplary embodiment of the invention, shown in part as a block diagram and in part as a circuit diagram;

FIG. 7 shows a sensor, modified as compared to FIG. 6, in accordance with another exemplary embodiment of the invention, shown in part as a block diagram and in part as a circuit diagram;

FIG. 8 shows a sensor for determining a value indicative of the impedance of a suspension according to a further exemplary embodiment of the invention in a side view; and

FIG. 9 shows an exemplary curve of permittivity values versus the excitation frequency and illustrates the derivation of characteristic properties of the suspension.

### DETAILED DESCRIPTION

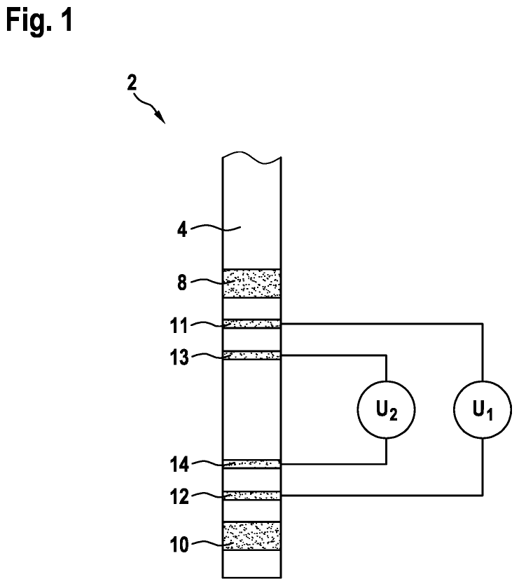

FIG. 1 shows a sensor 2 according to an exemplary embodiment of the invention in a side view. The sensor 2 is designed to determine a value indicative of the impedance of a suspension. The sensor 2 is designed for immersion in the suspension to be analyzed, in particular for immersion in a cell population to be analyzed. For this purpose, the sensor 2 has a rod-shaped sensor body 4, which is shown cut off in FIG. 1. The rod-shaped sensor body 4 can also be described as substantially cylindrical. The rod-shaped sensor body 4 may have a suitable length so that the analysis of the suspension can take place at a desired location in a container or reactor containing the suspension.

The rod-shaped sensor body 4 has six electrodes. In particular, the rod-shaped sensor body 4 has a pair of excitation electrodes, namely a first excitation electrode 8 and a second excitation electrode 10, a first pair of measurement electrodes, namely a first measurement electrode 11 and a second measurement electrode 12, and a second pair of measurement electrodes, namely a third measurement electrode 13 and a fourth measurement electrode 14. In the exemplary embodiment of FIG. 1, the six electrodes 8, 10, 11, 12, 13 and 14 are formed in a ring shape, i.e. they are formed circumferentially around the rod-shaped sensor body 4. It is emphasized that the six electrodes may also be present in other geometric configurations, for example in an elongated shape along the rod-shaped sensor body 4.

In the exemplary embodiment of FIG. 1, the six electrodes 8, 10, 11, 12, 13 and 14 are arranged in an end region of the rod-shaped sensor body 4. However, they may also be arranged in any other suitable region of the rod-shaped sensor body 4/of the sensor 2.

In the exemplary embodiment of FIG. 1, the first pair of measurement electrodes 11, 12 and the second pair of measurement electrodes 13, 14 are arranged between the excitation electrodes 8, 10. In particular, the first measurement electrode 11 is arranged adjacent to the first excitation electrode 8 and the third measurement electrode 13 is arranged adjacent to the first measurement electrode 11. Further in particular, the second measurement electrode 12 is arranged adjacent to the second excitation electrode 10 and the fourth measurement electrode 14 is arranged adjacent to the second measurement electrode 12. The second pair of measurement electrodes 13, 14 is arranged between the first pair of measurement electrodes 11, 12. The first measurement electrode 11 and the third measurement electrode 13 are closer to the first excitation electrode 8 than an imaginary centerline between the first excitation electrode 8 and the second excitation electrode 10. The second measurement electrode 12 and the fourth measurement electrode 14 are closer to the second excitation electrode 10 than an imaginary centerline between the first excitation electrode 8 and the second excitation electrode 10. By arranging the measurement electrodes 11, 12, 13, 14 between the excitation electrodes 8, 10 and in the vicinity of the excitation electrodes 8, 10, a comparatively high voltage can be measured when the excitation current is applied.

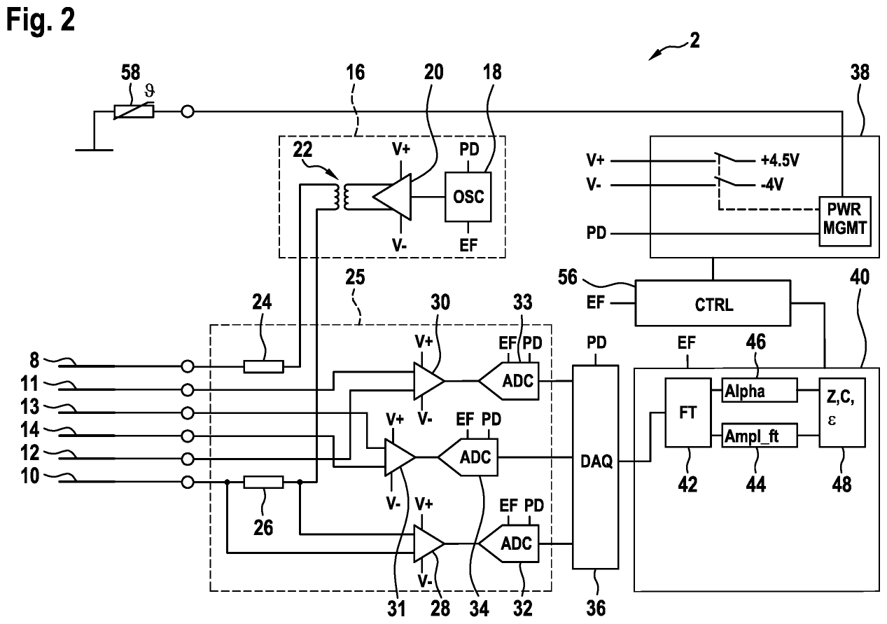

In operation, a first voltage Ui is measured between the first pair of measurement electrodes 11, 12 and a second voltage U2 is measured between the second pair of measurement electrodes 13, 14. This is shown schematically in FIG. 1 and will be described in detail below with reference to FIG. 2.

FIG. 2 shows a sensor 2 according to an exemplary embodiment of the invention, shown in part as a block diagram and in part as a circuit diagram. The components of the sensor 2 of FIG. 2 may be present in a sensor having the physical shape shown in FIG. 1. That is, the circuitry or signal processing structure of the sensor 2 of FIG. 2 may be the structure of the electrical components of the sensor 2 of FIG. 1. The components shown in FIG. 2 to the right of the circularly drawn coupling points can be accommodated in the sensor body 4 or in a component adjoining the same.

The sensor 2 comprises the first excitation electrode 8, the second excitation electrode 10, the first measurement electrode 11, the second measurement electrode 12, the third measurement electrode 13, and the fourth measurement electrode 14 described above. The six electrodes 8, 10, 11, 12, 13, 14 are accessible from the outside, i.e. they are in contact with the suspension when the sensor 2 is immersed in the suspension for analysis of the suspension. Furthermore, a temperature sensor 58 is provided, which is located outside the housing of the sensor 2.

The sensor 2 has an oscillator circuit 16, a signal detection and processing circuit 25, a data storage 36, a data processing device 40, a control unit 56, and a power management unit 38. The individual components and operation of these subsystems will be described in detail in the following.

The oscillator circuit 16 comprises an oscillator 18 coupled to an oscillation amplifier 20, which in turn is coupled to a transformer 22. The oscillator 18 is supplied with the desired excitation frequency EF via a control input. The excitation frequency EF is determined by the control unit 56, as described in detail below, and supplied to the oscillator 18. The oscillator 18 generates an oscillation having the excitation frequency EF, which is passed to the oscillation amplifier 20. The oscillation amplifier 20 generates an excitation current with the excitation frequency EF through the primary winding of the transformer 22. By induction, the excitation current is transferred to the secondary winding of the transformer 22, from where the current is applied to the first and second excitation electrodes 8, 10. One end of the secondary winding is connected to the first excitation electrode 8 via a first resistor 24 and the second end of the secondary winding is connected to the second excitation electrode 10 via a second resistor 26. Thus, there is formed a closed circuit from the first end of the secondary winding through the first resistor 24, via the first excitation electrode 8 through the suspension to the second excitation electrode 10, and through the second resistor 26 to the second end of the secondary winding. In this manner, an excitation current oscillating at the excitation frequency EF is generated through the suspension between the first excitation electrode 8 and the second excitation electrode 10. In the exemplary embodiment of FIG. 2, the excitation current is a sinusoidal excitation current oscillating at the excitation frequency EF. Further, in the exemplary embodiment of FIG. 2, the excitation current has an amplitude of 1 Vpp to 2 Vpp.

The transformer 22 provides for galvanic decoupling between the oscillation amplifier 20 and the first and second excitation electrodes 8, 10. A coupling capacitance may be provided in parallel with the transformer 22. The transformer 22 may then be said to have a parallel capacitance, which is present between the primary winding and the secondary winding. The parallel capacitance may be a discrete component or a parasitic capacitance of the transformer. The parallel capacitance, by being arranged parallel to the transformer, can counteract interfering influences from other coupling capacitances, such as coupling capacitances between the electrodes and the container of the suspension and/or coupling capacitances between the electrodes and other sensors present in the suspension. The parallel capacitance may be between 1 pF and 5 pF.

The excitation current between the first excitation electrode 8 and the second excitation electrode 10, oscillating at the excitation frequency EF, produces a first AC voltage between the first measurement electrode 11 and the second measurement electrode 12, and a second AC voltage between the third measurement electrode 13 and the fourth measurement electrode 14. Both the excitation current and the first voltage between the first pair of measurement electrodes 11, 12 and the second voltage between the second pair of measurement electrodes 13, 14 are detected and sampled by the signal detection and processing circuit 25. At the end of the signal processing in the signal detection and processing circuit 25, there are digital signals for the excitation current, the first voltage and the second voltage.

A first signal representing the excitation current is obtained in the following manner. The second resistor 26 acts as a measuring resistor for the excitation current. The voltage across the measuring resistor 26 is tapped by means of two conductors and supplied as the first signal to a first amplifier circuit 28. The amplified first signal is fed to the first analog-to-digital converter 32. There, the amplified first signal is converted into a digital signal, i.e. the amplified first signal is sampled and quantized. The resulting first sampling values are output to the data storage 36. It can be seen that the first resistor 24 is not required for obtaining the first signal. However, for reasons of symmetry, the first resistor 24 is provided nevertheless. Furthermore, it can be seen that tapping of a signal representing the excitation current can also take place at the first resistor 24. That is, the first amplifier circuit could also be coupled to the first excitation electrode 8 or the first resistor 24. For example, the first resistor 24 and the second resistor 26 may each have a value from 30Ω to 50Ω.

The voltage between the first measurement electrode 11 and the second measurement electrode 12 forms a second signal, which is fed to a second amplifier circuit 30. There, the second signal is amplified, and the amplified second signal is fed to a second analog-to-digital converter 33. The second analog-to-digital converter 33 generates, analogously to the first analog-to-digital converter 32, second sampling values which are discrete in time and quantized. The second sampling values are also output to the data storage 36.

The voltage between the third measurement electrode 13 and the fourth measurement electrode 14 forms a third signal, which is fed to a third amplifier circuit 31. There, the third signal is amplified, and the amplified third signal is supplied to a third analog-to-digital converter 34. The third analog-to-digital converter 34 generates, analogously to the first analog-to-digital converter 32, third sampling values io which are discrete in time and quantized. The third sampling values are also output to the data storage 36.

The first analog-to-digital converter 32, the second analog-to-digital converter 33, and the third analog-to-digital converter 34 also receive the information on the excitation frequency EF from the control unit 56. The first analog-to-digital converter 32, the second analog-to-digital converter 33 and the third analog-to-digital converter 34 use 4 times the excitation frequency EF for sampling the amplified first signal, the amplified second signal, and the amplified third signal. Thus, the first analog-to-digital converter 32, the second analog-to-digital converter 33, and the third analog-to-digital converter 34 generate first, second, and third sampling values for the excitation current, the first voltage, and the second voltage using 4 times the excitation frequency EF.

The first, second and third sampling values, output by the first analog-to-digital converter 32, the second analog-to-digital converter 33 and the third analog-to-digital converter 34, are temporarily stored or buffered in the data storage 36. Thus, the data storage 36 constitutes a buffer that holds the first sampling values, the second sampling values, and the third sampling values and can make them available for further data processing independent of real-time. Thus, from the data storage 36 onward, there are no longer any real-time requirements for the down-stream components. On the contrary, the downstream components can access a database accumulated over a period of time in the data storage 36. The data storage 36 may be, for example, a DPRAM or any other suitable type of data storage.

The data storage 36 is coupled to the data processing device 40 and outputs the first sampling values for the excitation current, the second sampling values for the first voltage between the first measurement electrode 11 and the second measurement electrode 12, and the third sampling values for the second voltage between the third measurement electrode 13 and the fourth measurement electrode 14 to the data processing device 40.

In the data processing device 40, the first, second and third sampling values are transferred to a Fourier transform module 42. The Fourier transform module 42 performs two discrete, complex Fourier transforms on the sampling values. In particular, the Fourier transform module 42 performs a first discrete complex Fourier transform with the first sampling values for the excitation current and the second sampling values for the voltage between the first measurement electrode 11 and the second measurement electrode 12, i.e., with the sampling values for the excitation current and the sampling values for the first voltage. Further in particular, the Fourier transform module 42 performs a second discrete complex Fourier transform with the first sampling values for the excitation current and the third sampling values for the voltage between the third measurement electrode 13 and the fourth measurement electrode 14, i.e. with the sampling values for the excitation current and the sampling values for the second voltage.

The Fourier transforms performed in the Fourier transform module 42 are discrete and complex, because the time-discrete sampling values for the excitation current and for the respective voltage are analyzed as interdependent quantities. The result of these complex Fourier transforms are the amplitudes of the excitation current and the respectively measured voltage for different frequencies as well as the phase shift a between the excitation current and the respectively measured voltage for the various frequencies. It is possible that the Fourier transforms perform a broad spectral analysis of the sampling values and that all spectral components except for the spectral components at the excitation frequency EF are discarded then. However, it is also possible for the Fourier transforms specifically determine the spectral component of the excitation current as well as the spectral component of the respective voltage between the respective measurement electrodes at the excitation frequency. In this context, the Goertzel algorithm can also be used to specifically determine the spectral components at the excitation frequency EF.

The amplitude of the spectral component of the excitation current at the excitation frequency EF and the amplitude of the spectral component of the respective measured voltage at the excitation frequency EF are passed to an impedance and permittivity determination module 48 via a first data transmission link 44. The phase shift a between the spectral component of the excitation current at the excitation frequency and the spectral component of the respective measured voltage at the excitation frequency is transferred to the impedance and permittivity determination module 48 via a second data transmission link 46.

The impedance and permittivity determination module 48 determines from the transferred parameters a first impedance measurement value Z_{sig}|₁, a second impedance measurement value Z_{sig}|₂, an impedance value Z, a capacitance value C and the permittivity c of the suspension at the excitation frequency. The first impedance measurement value Z_{sig}|₁ is obtained from the amplitude of the excitation current at the excitation frequency EF, the amplitude of the first voltage at the excitation frequency EF, and the phase shift a between the excitation current and the first voltage at the excitation frequency EF. The first impedance measurement value Z_{sig}|₁ is thus a complex impedance measurement value at the excitation frequency EF. The second impedance measurement value Z_{sig}|₂ is obtained from the amplitude of the excitation current at the excitation frequency EF, the amplitude of the second voltage at the excitation frequency EF and the phase shift a between excitation current and second voltage at the excitation frequency EF. The second impedance measurement value Z_{sig}|₂ is thus a complex impedance measurement value at the excitation frequency EF. As described above, the afore-mentioned amplitudes of excitation current, first voltage and second voltage as well as the afore-mentioned phase shifts are available as results of the first and second complex Fourier transforms. Thus, the first impedance measurement value Z_{sig}|₁ and the second impedance measurement value Z_{sig}|₂ can be conveniently calculated from the data present in the impedance and permittivity determination module 48.

It is emphasized that the first impedance measurement value Z_{sig}|₁ and the second impedance measurement value Z_{sig}|₂ may also be determined in other ways from the first signal, i.e., the voltage tapped at the second resistor 26, the second signal, i.e., the voltage tapped at the first pair of measurement electrodes 11, 12, and the third signal, i.e., the voltage tapped at the second pair of measurement electrodes 13, 14. Although the signal processing described in detail above permits a particularly accurate determination of the first impedance measurement value Z_{sig}|₁ and the second impedance measurement value Z_{sig}|₂, the manner of determining the first impedance measurement value Z_{sig}|₁ and the second impedance measurement value Z_{sig}|₂ is not decisive for the determination of the impedance value Z described below. In this respect, any suitable kind of signal processing can be used.

The impedance and permittivity determination module 48 determines the impedance value Z according to the following formula:

$${Z = k \frac{1}{( {λ}_{1} - {λ}_{2 } )} {( {G}_{el}^{- 1} ( {Z}_{sig} ) \vert}_{1} - {G}_{el}^{- 1} ( {Z}_{sig} ) \vert}_{2} ) ,$$

wherein Z_{sig}|₁ denotes the first impedance measurement value and Z_{sig}|₂ denotes the second impedance measurement value, as described above. Gₑₗ⁻¹ denotes a correction function that represents the transmission behavior of the measurement arrangement. In the exemplary embodiment of FIG. 2, Gₑₗ⁻¹ corrects those artifacts that have been introduced into the signals between the respective electrodes and the associated analog-to-digital converters. These may include, for example, propagation time differences in the individual signal paths, non-linear gains in the amplifier circuits, etc. Accordingly, Gₑₗ⁻¹ fully or at least approximately restores those impedance measurement values that have been applied directly to the electrodes. Furthermore, λ₁ denotes a first geometry factor representing the measurement geometry of the first pair of measurement electrodes 11, 12, and λ₂ denotes a second geometry factor representing the measurement geometry of the second pair of measurement electrodes 13, 14. The first geometry factor λ₁ and the second geometry factor λ₂ describe the respective measurement cells underlying the voltage measurements at the first pair of measurement electrodes 11, 12 and at the second pair of measurement electrodes 13, 14. The nature of the first geometry factor λ₁ and the second geometry factor λ₂ will be described in detail further below. The variable k denotes a proportionality constant.

By using the first impedance measurement value Z_{sig}|₁ and the second impedance measurement value Z_{sig}|₂ and correlating the first impedance measurement value Z_{sig}|₁ and the second impedance measurement value Z_{sig}|₂ according to the above formula, an impedance value Z can be determined that is very robust with respect to interferences. In particular, by determining the difference between the impedance measurement values adjusted by way of the correction function Gₑₗ⁻¹ and by determining the difference between the geometry factors, it can be achieved that low-order interferences in the two measurements cancel each other out. The impedance value Z, obtained by the above formula, contains only higher order interferences, which are comparatively small in many applications. Thus, high measurement accuracy can be achieved.

The capacitance value C and the permittivity c are then calculated from the impedance value Z. In this way, the material property permittivity ε of the suspension is derived from the impedance value Z as a result. For deriving the permittivity c, perse known approaches and methods can be used. By obtaining the impedance value Z with high accuracy, as described herein, improved results can be obtained, compared to previous sensors, even when using known methods for deriving the permittivity ε.

Thus, the impedance value Z, the capacitance value C, and the permittivity c are available as results of the signal processing in the data processing device 40. In particular, these values are available as results for the excitation of the suspension with a specific excitation frequency. One or more of these values may be output for further processing. The output may be made to an external unit or, as shown in the exemplary embodiment of FIG. 2, to the control unit 56 provided in the sensor 2. In the exemplary embodiment of FIG. 2, the value determined for the permittivity ε is output to the control unit 56.

The data processing device 40 may be implemented in software or may be an arrangement of hardware components. It is also possible that the data processing device 40 is implemented partly in software and partly in hardware. The same applies to the control unit 56 described below.

The control unit 56 is connected to the power management unit 38, to the oscillator circuit 16, to the signal detection and processing circuit 25, and to the data processing device 40. The control unit 56 controls the method for determining a value indicative of the impedance of a suspension in accordance with exemplary embodiments of the invention. For this purpose, the control unit 56 is arranged to specify the excitation frequency EF for the method. In particular, the control unit is arranged to successively determine a plurality of excitation frequencies for a plurality of runs of the method in the framework of an impedance spectroscopy.

For a given run, the control unit 56 transmits the specified excitation frequency EF to the oscillator circuit 16, where the oscillator 18 generates an oscillation at the excitation frequency EF, to the signal detection and processing circuit 25, where the first analog-to-digital converter 32, the second analog-to-digital converter 33 and the third analog-to-digital converter 34 adjust the sampling rate based on the excitation frequency EF, and to the data processing device 40 where the Fourier transform module 42 analyzes the sampling values of excitation current, first voltage and second voltage with respect to the spectral signal components at the excitation frequency.

Further, in the exemplary embodiment of FIG. 2, the control unit 56 is coupled to the data processing device 40 in so far as the data processing device 40 transmits the permittivity ε, determined for the excitation frequency EF, to the control unit 56. The control unit 56 can then determine a new excitation frequency for the next run of the method in the framework of an impedance spectroscopy.

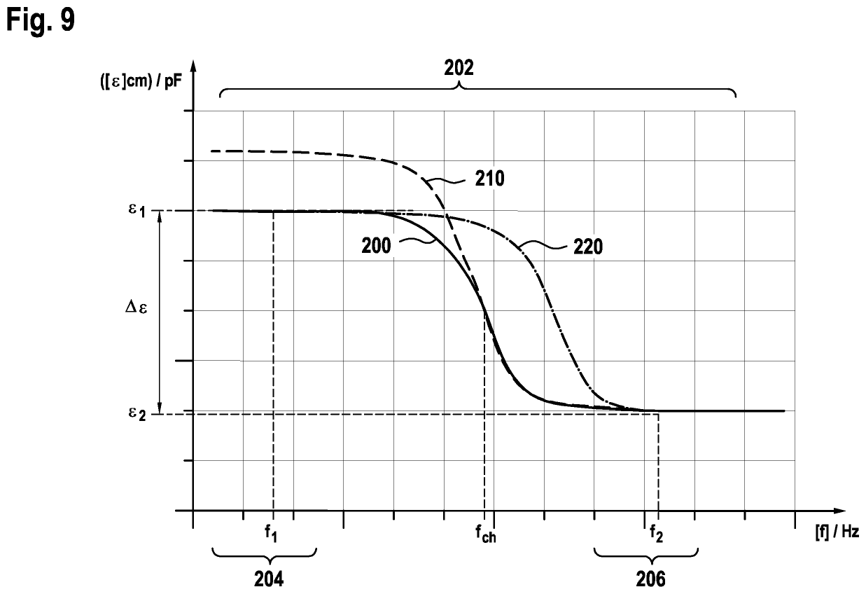

After determining a plurality of permittivity values for different excitation frequencies, the control unit 56 may derive one or more characteristic properties of the suspension from the plurality of permittivity values. To this end, the control unit may plot a curve through the plurality of permittivity values and derive the characteristic properties of the suspension from the curve, as described below with reference to FIG. 9. Such correlation of the plurality of permittivity values may also be performed externally of the sensor 2.

The control unit 56 is coupled to the power management circuit 38 in order to signal the start and end of a run of the method. Based on these signals, the power management circuit 38 supplies the oscillation amplifier 20 as well as the first amplifier circuit 28, the second amplifier circuit 30 and the third amplifier circuit 31 with the positive supply voltage V+ and the negative supply voltage V−, which are +4.5 V and −4 V in the present exemplary embodiment. At the end of a run of the method, the power management circuit 38 disconnects the positive and negative supply voltages and transmits a power down signal (“power down”) to the oscillator 18, the first analog-to-digital converter 32, the second analog-to-digital converter 33, the third analog-to-digital converter 34, and the data storage 36. In this manner, the sensor may conserve electrical energy between the runs of the method for determining the value indicative of the impedance.

The power management circuit 38 may obtain the electrical energy from outside the sensor 2 or via an internal energy reservoir, such as in the form of a battery.

To protect the sensor 2, the power management circuit 38 may open the voltage supply when the temperature sensor 58 measures a temperature above a predetermined threshold.

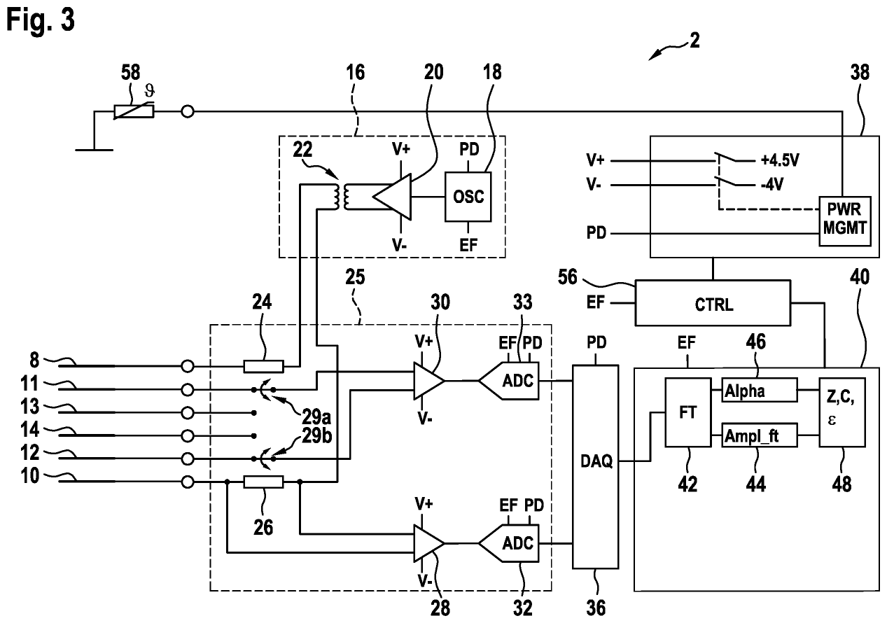

FIG. 3 shows a sensor 2 according to a further exemplary embodiment of the invention that is modified with respect to FIG. 2, again shown in part as a block diagram and in part as a circuit diagram. In particular, the modification relates to the signal processing in the signal detection and processing circuit 25. In general, corresponding components are provided with the same reference numerals as in FIG. 2. For their description, reference is made to the above explanations.

The signal detection and processing circuit 25 of the embodiment of FIG. 3 has no third amplifier circuit 31 and no third digital-to-analog converter 34. Instead, the second amplifier circuit 30 can be selectively connected to the first pair of measurement electrodes 11, 12 or to the second pair of measurement electrodes 13, 14. A first selection switch 29a and a second selection switch 29b are provided for this purpose. The first selection switch 29a connects either the first measurement electrode 11 or the third measurement electrode 13 to the second amplifier circuit 30. The second selection switch 29b connects either the second measurement electrode 12 or the fourth measurement electrode 14 to the second amplifier circuit 30. Thus, either the first voltage, i.e., the voltage between the first and second measurement electrodes 11, 12, or the second voltage, i.e., the voltage between the third and fourth measurement electrodes 13, 14, can be passed on to the second analog-to-digital converter 33 via the second amplifier circuit 30.

For the method for determining the value indicative of the impedance of the suspension, the modification of FIG. 3 means that the first impedance measurement value and the second impedance measurement value are determined on the basis of signals which are tapped with a time offset from each other. However, the method can be implemented with a sensor 2 having only one further amplifier circuit 30 in addition to the first amplifier circuit 28 and only one further analog-to-digital converter 33 in addition to the first analog-to-digital converter 32.

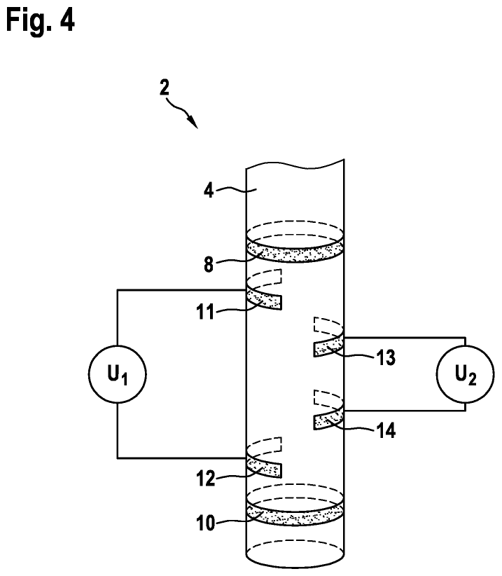

FIG. 4 shows a sensor 2 according to a further exemplary embodiment of the invention, modified with respect to FIG. 1, in a perspective view. The modification relates to the geometric arrangement of the first and second pairs of measurement electrodes. Compared to the embodiment of FIG. 1, the first measurement electrode 11, the second measurement electrode 12, the third measurement electrode 13 and the fourth measurement electrode 14 are not formed in a ring shape, but are formed in a partial ring shape. Each of the four measurement electrodes extends across a circular sector of slightly less than 180° along the cylindrical outer surface of the rod-shaped sensor body 4. In the view of FIG. 4, the first measurement electrode 11 and the second measurement electrode 12 are arranged on the left side of the sensor body 4, and the third measurement electrode 13 and the fourth measurement electrode 14 are arranged on the right side of the sensor body 4. In other words, the first pair of measurement electrodes 11, 12 and the second pair of measurement electrodes 13, 14 are arranged on different sides of the sensor 2. Such an arrangement allows a low mutual influence of the pairs of electrodes on each other, which could have a potentially negative effect on the measurement accuracy.

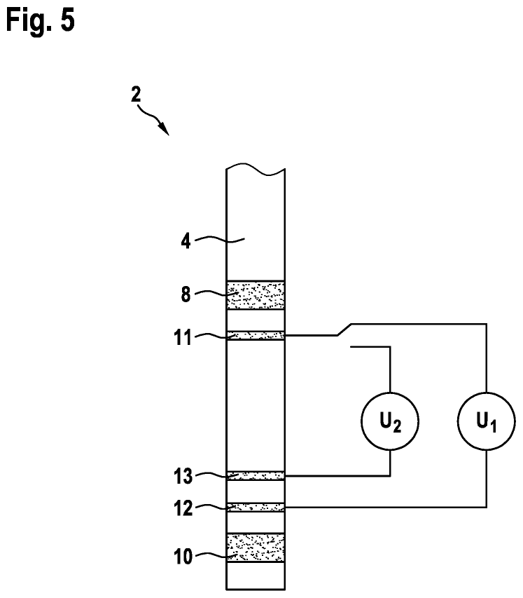

FIG. 5 shows a sensor 2 according to a further exemplary embodiment of the invention, modified with respect to FIG. 1, in a side view. The modification relates to the number and the geometric arrangement of the measurement electrodes. The sensor 2 of the exemplary embodiment of FIG. 5 has three measurement electrodes, a first measurement electrode 11, a second measurement electrode 12 and a third measurement electrode 13. The first measurement electrode 11 and the second measurement electrode 12 correspond in their arrangement to the first pair of measurement electrodes 11, 12 of the embodiment of FIG. 1. The third measurement electrode 13 of the embodiment of FIG. 5 is arranged where the fourth measurement electrode 14 was arranged in the embodiment of FIG. 1.

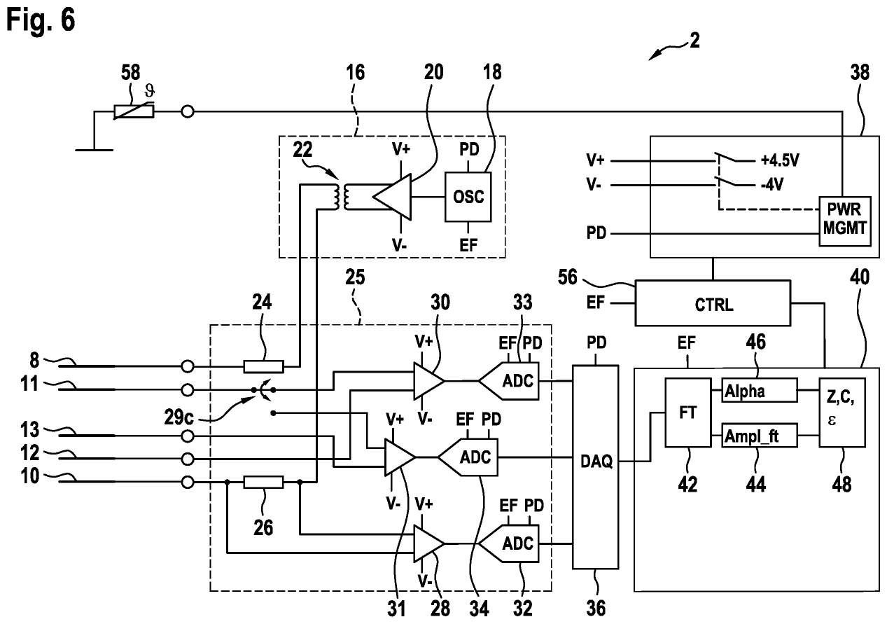

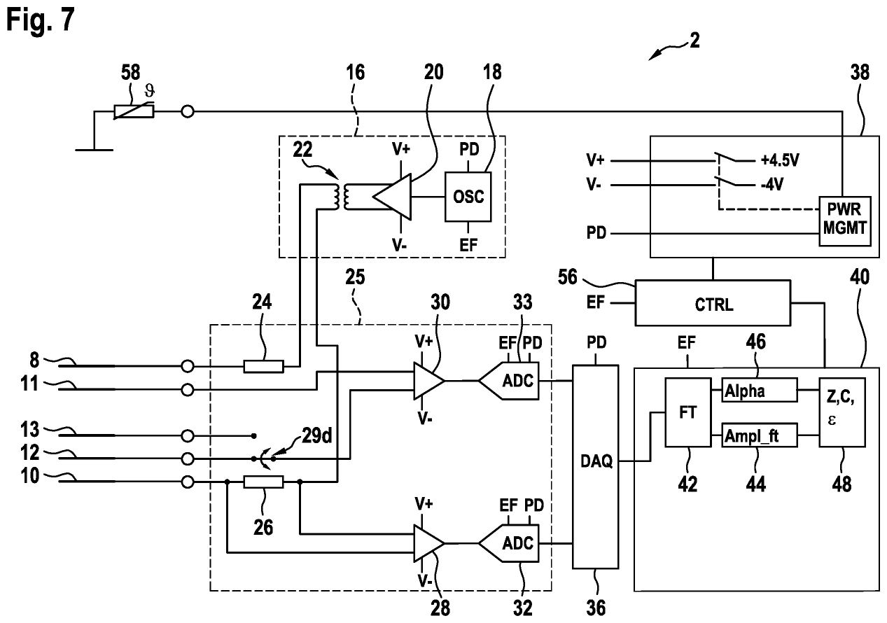

In the exemplary embodiment of FIG. 5, the first pair of measurement electrodes consists of the first measurement electrode 11 and the second measurement electrode 12. The second pair of measurement electrodes consists of the first measurement electrode 11 and the third measurement electrode 13. In operation, a first voltage U₁ is measured at the first pair of measurement electrodes 11, 12, whereas a second voltage U₂ is measured at the second pair of measurement electrodes 11, 13. In other words, the first measurement electrode 11 forms a potential reference point for both the measurement of the first voltage and the measurement of the second voltage. Two exemplary embodiments of the downstream signal processing of the sensor 2 of FIG. 5 are described below with reference to FIGS. 6 and 7.

FIG. 6 shows a sensor 2 according to a further exemplary embodiment of the invention, shown in part as a block diagram and in part as a circuit diagram. The components of the sensor 2 of FIG. 6 may be present in a sensor of the physical configuration shown in FIG. 5. That is, the circuitry or signal processing structure of the sensor 2 of FIG. 6 may be the structure of the electrical components of the sensor 2 of FIG. 5. Thus, FIG. 6 relates to FIG. 5 in the same way as FIG. 2 to FIG. 1. The sensor 2 of FIG. 6 is overall very similar and in large parts identical to the sensor 2 of FIG. 2. Corresponding components are provided with corresponding reference numerals. For the description of the components, reference is made to the description of FIG. 2 above.

The changes in the embodiment of FIG. 6 with respect to the embodiment of FIG. 2 take into account the fact that the sensor 2 of FIG. 6 has only three measurement electrodes 11, 12 and 13, as explained above with reference to FIG. 5. The first measurement electrode 11 can be selectively connected to the second amplifier circuit 30 or the third amplifier circuit 31 by means of a selection switch 29c. The second measurement electrode 12 is connected to the second amplifier circuit 30, and the third measurement electrode 13 is connected to the third amplifier circuit 31. Thus, either the first voltage can be supplied to the second analog-to-digital converter 33 via the second amplifier circuit 30, or the second voltage can be supplied to the third analog-to-digital converter 34 via the third amplifier circuit 31. For the method for determining the value indicative of the impedance of the suspension, this means that the first impedance measurement value and the second impedance measurement value are determined on the basis of signals which are tapped with a time offset from each other.

FIG. 7 shows a sensor 2 according to a further exemplary embodiment of the invention, modified with respect to FIG. 6, again shown in part as a block diagram and in part as a circuit diagram. In particular, the modification relates to the signal processing in the signal detection and processing circuit 25. In general, corresponding components are provided with the same reference numerals as in FIG. 6. For their description, reference is made to the above explanations.

The signal detection and processing circuit 25 of the embodiment of FIG. 7 has no third amplifier circuit 31 and no third digital-to-analog converter 34. Instead, the second amplifier circuit 30 is permanently connected to the first measurement electrode 11 and can be selectively connected to the second measurement electrode 12 or the third measurement electrode 13. A selection switch 29d is provided for this purpose. Thus, via the second amplifier circuit 30, either the first voltage, i.e. the voltage between the first and second measurement electrodes 11, 12, or the second voltage, i.e. the voltage between the first and third measurement electrodes 11, 13, can be passed on to the second analog-to-digital converter 33. For the method for determining the value indicative of the impedance of the suspension, this means that the first impedance measurement value and the second impedance measurement value are determined on the basis of signals which are tapped with a time offset from each other.

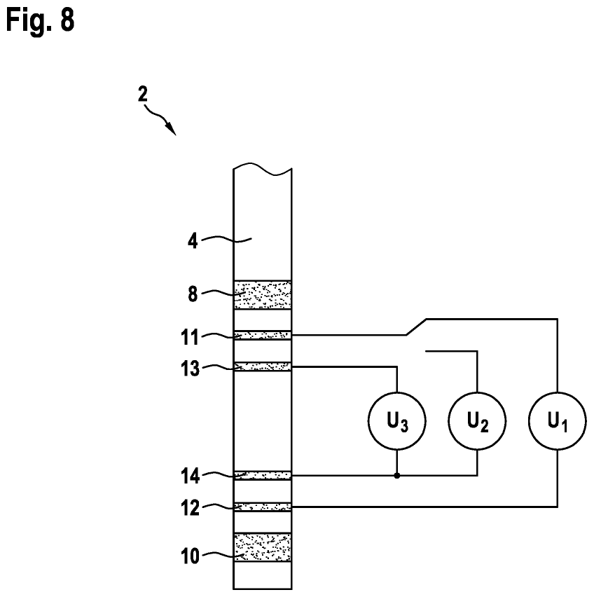

FIG. 8 shows a sensor 2 according to a further exemplary embodiment of the invention in a side view. Like the sensor 2 of FIG. 1, the sensor 2 of FIG. 8 has a first excitation electrode 8, a second excitation electrode 10, a first measurement electrode 11, a second measurement electrode 12, a third measurement electrode 13 and a fourth measurement electrode 14. The six electrodes are arranged in the sensor 2 of FIG. 8 in the same way as in the sensor 2 of FIG. 1. However, the four measurement electrodes 11, 12, 13, 14 of the sensor 2 of FIG. 8 form three pairs of measurement electrodes. In particular, a first pair of measurement electrodes consists of the first measurement electrode 11 and the second measurement electrode 12. A second pair of measurement electrodes consists of the first measurement electrode 11 and the fourth measurement electrode 14. A third pair of measurement electrodes consists of the third measurement electrode 13 and the fourth measurement electrode 14.

In operation, a first voltage U₁ is measured at the first pair of measurement electrodes 11 and 12, a second voltage U₂ is measured at the second pair of measurement electrodes 11 and 14, and a third voltage U₃ is measured at the third pair of measurement electrodes 13 and 14. On the basis of the excitation current, the first voltage U₁, the second voltage U₂, and the third voltage U₃, a first impedance measurement value, a second impedance measurement value, and a third impedance measurement value are determined. These three impedance measurement values are correlated with each other to determine an impedance value for the suspension. By using three pairs of measurement electrodes, which are created from a total of four measurement electrodes, interferences can be eliminated particularly well and a particularly accurate impedance value for the suspension can be determined.

The impedance value Z can be determined according to the following formula:

$${Z}^{2 } = {k}_{2} \frac{{{{λ}_{3} ( {G}_{el}^{- 1} ( {Z}_{sig} ) \vert}_{2} - {G}_{el}^{- 1} ( {Z}_{sig} ) \vert}_{1} )}{( {λ}_{1} - {λ}_{2} ) ( {λ}_{1} - {λ}_{3} ) ( {λ}_{2} - {λ}_{3} )} ++ {k}_{2} \frac{{{{λ}_{2} ( {G}_{el}^{- 1} ( {Z}_{sig} ) \vert}_{1} - {G}_{el}^{- 1} ( {Z}_{sig} ) \vert}_{3} )}{( {λ}_{1} - {λ}_{2} ) ( {λ}_{1} - {λ}_{3} ) ( {λ}_{2} - {λ}_{3} )} ++ {k}_{2} \frac{{{{λ}_{1} ( {G}_{el}^{- 1} ( {Z}_{sig} ) \vert}_{3} - {G}_{el}^{- 1} ( {Z}_{sig} ) \vert}_{2} )}{( {λ}_{1} - {λ}_{2} ) ( {λ}_{1} - {λ}_{3} ) ( {λ}_{2} - {λ}_{3} )} ,$$

wherein Z_{sig}|₁ denotes the first impedance measurement value, Z_{sig}|₂ denotes the second impedance measurement value and Z_{sig}|₃ denotes the third impedance measurement value. Gₑₗ⁻¹ denotes a correction function that represents the transmission behavior of the measurement arrangement, as described above. Furthermore, λ₁ denotes a first geometry factor representing the measurement geometry of the first pair of measurement electrodes 11, 12, λ₂ denotes a second geometry factor representing the measurement geometry of the second pair of measurement electrodes 11, 14, and λ₃ denotes a third geometry factor representing the measurement geometry of the third pair of measurement electrodes 13, 14. The variable k₂ denotes a proportionality constant.

The explanations given above with respect to FIGS. 2, 3, 6 and 7 concerning simultaneous or time-offset signal processing apply analogously to the sensor 2 of FIG. 8. For example, the four measurement electrodes can be coupled to two amplifier circuits and two analog-to-digital converters, so that partially simultaneous and partially time-offset signal processing takes place for the three measuring voltages. For example, the first voltage U₁ and the third voltage U₃ may be measured substantially simultaneously, while the second voltage U₂ is measured thereafter. It is also possible, for example, that the four measurement electrodes are coupled to a single amplifier circuit and a single analog-to-digital converter by means of suitable selection switches, and the three voltages are measured one after the other.

It is emphasized furthermore that more than four measurement electrodes may be present and more than three pairs of measurement electrodes may be formed. Also, more than three impedance measurement values can be correlated for determining the value indicative of the impedance of the suspension. Two formulas are given above by which it is possible to determine a value indicative of the impedance of the suspension in the case of two impedance measurement values and in the case of three impedance measurement values. Some understanding aids to the formulas are provided below.

As described above, the objective of the method is to determine a value indicative of the impedance of a suspension. This value can be, for example, directly the impedance value Z.

In a real measurement setup, an impedance measurement value Z_{sig} may be determined from a measured voltage U_{sig} and measured current I_{sig}. However, this impedance measurement value may differ from the impedance value Zₘₑₛₛ present at the measurement electrodes due to the signal processing in the measurement arrangement. The conversion between the measured impedance value Z_{sig} and the impedance value Zₘₑₛₛ present at the measurement electrodes can be expressed by a transfer function Gₑₗ of the measurement arrangement or a correction function Gₑₗ⁻¹ inverse to the same:

$$\begin{matrix} & {G}_{el} & \\ {Z}_{sig} & ⇄ & {Z}_{mess} \\ & {G}_{el}^{- 1} & \end{matrix} .$$

In reality, the impedance value Zₘₑₛₛ present at the measurement electrodes is not the sought impedance of the suspension, but is composed of an impedance value Z_{c.c.}, which depends on the measurement geometry, and a large number of interfering influences, such as parasitic capacitances, double layer formation on the electrode surfaces, contact resistances, etc. The interfering influences can be collectively referred to as parasitic influences Zₚₐᵣ which distort the measurement. The totality of the parasitic influences can depend on a variety of parameters, such as temperature, conductivity of the solution, frequency of the excitation current, ion concentration in the solution, material and nature of the electrodes, etc. One can abbreviate the notation for these parameters to a parameter set {ψᵢ}. The parameters can contribute in different ways to Zₚₐᵣ and thus be designated as different Zⁱ⁻ₚₐᵣ({ψᵢ}). Thus, the impedance value Zₘₑₛₛ present at the measurement electrodes can be expressed as a function F according to the following relationship:

$$\begin{matrix} & {G}_{el} & \\ {Z}_{sig} & ⇄ & F ( {Z}_{par}^{i} ( \{ {ψ}_{i} \} ) , {Z}_{c . c .} ) . \\ & {G}_{el}^{- 1} & \end{matrix}$$

The term Z_{c.c.} is used because the impedance of a cell suspension can be described in a good approximation by the so-called Cole-Cole impedance.

As described above, the impedance value Z_{c.c.} depends on the measurement geometry. The measurement geometry is also referred to herein as the geometry of the measurement cell of a pair of measurement electrodes. The impedance value Z sought is related to the measurement geometry dependent impedance value Z_{c.c.} via the cell constant λ. The following relationship applies:

$${Z}_{c . c .} = \frac{{λ}_{j}}{i {ωϵ}_{cole \text{-} cole}} = {λ}_{j} Z .$$

For the cell constant λⱼ of a j^{th} pair of measurement electrodes holds:

$${λ}_{j} = \frac{1}{{σ}_{solution} Δ {ϕ}^{[ j ]}} \int \vec{J} d \vec{A} ,$$

wherein

∫\vec{J}d\vec{A}

is the area integral of the current densities and Δϕ^{(j)} is the potential difference of the j^{th} pair of measurement electrodes.

Thus, the impedance value Zₘₑₛₛ present at the measurement electrodes can be expressed according to the following relationship:

$$\begin{matrix} & {G}_{el} \\ {Z}_{sig} & ⇄ \\ & {G}_{el}^{- 1}\end{matrix} F ( {Z}_{par}^{i} ( \{ {ψ}_{i} \} ) , {λ}_{j} Z ) .$$

The function F can be expanded as polynomial of λⱼZ. Thus, the result is:

$$\begin{matrix} & {G}_{el} \\ {Z}_{sig} & ⇄ \\ & {G}_{el}^{- 1}\end{matrix} {\sum }_{n} \frac{1}{n !} {a}_{n} ( {Z}_{par}^{i} ( \{ {ψ}_{i} \} ) ) {λ}_{j}^{a} {Z}^{n} .$$

For the j^{th} pair of measurement electrodes, the second order expansion results in:

Gₑₗ⁻¹(Z_{sig})|ⱼ=a₀((Zₚₐᵣⁱ))+λⱼa₁((Zₚₐᵣⁱ))Z+O(2),

wherein O(2) is an abbreviation for the quadratic part of the polynomial expansion.

If one assumes that the prefactors aₙ and the parasitic influences Zⁱₚₐᵣ are identical for different cell geometries, and if one assumes furthermore a series connection of parasitic influences and cell impedance, the difference for two impedance measurement values results as follows:

Gₑₗ^{'11}(Z_{sig})|₁−Gₑₗ⁻¹(Z_{sig})|₂=(λ₁−λ₂)a₁((Zₚₐᵣⁱ))Z+O(2),

wherein O(2) is an abbreviation for all quadratic parts of the polynomial expansions.

If one further assumes that O(2) as well as the portions of still higher order are negligible and that a₁({Zₚₐᵣⁱ}) in good approximation is a constant, one arrives at the above formula for the calculation of the value Z indicative of the impedance of the suspension from two impedance measurement values.

It has been found that under the assumptions described above, measurement results of very high accuracy are possible.

A further development of the above considerations leads to the additional above-mentioned formula for calculating the value Z indicative of the impedance of the suspension from three impedance measurement values.

FIG. 9 shows, purely qualitatively, the course 200 of the permittivity c of a cell population, plotted against the excitation frequency f. The course 200 is a purely exemplary curve derived from a plurality of permittivity values determined by the method described above. For example, the course 200 may have been derived from the plurality of permittivity values by means of a Cole-Cole fitting.

Characteristics of the cell population can be derived from the course 200 as follows. In FIG. 9, it is qualitatively shown that upstream of a frequency f_{ch}, characteristic of the β-dispersion region 202, is a plateau region 204 in which the permittivity ε, compared to the region around the characteristic frequency f_{ch}, changes only little with frequency, and that downstream of the characteristic frequency f_{ch}, another plateau region 206 is located, which is different from the plateau region 204 upstream of the characteristic frequency f_{ch} and in which the permittivity ε, again compared to the region around the characteristic frequency f_{ch}, also does not change much with frequency.

Comparing a permittivity value ε₁ representing the permittivity ε at an excitation frequency f₁ in the plateau region 204 with a permittivity value ε₂ at an excitation frequency f₂ in the plateau region 206, a difference value Δε of the two permittivity values can be determined from the permittivity values ε₁ and ε₂ determined at the excitation frequencies f₁ and f₂, respectively. The difference value Δε is a measure for the number of living cells contained in the cell population. The alternative permittivity curve 210, indicated by two dots and three dashes, would result in an Δε at the respective excitation frequencies f₁ and f₂ that is larger in amount, permitting the conclusion that the cell population, for which the permittivity curve 210 was obtained, has more living cells in the same volume than the cell population underlying the permittivity curve 200.

A change in the characteristic frequency f_{ch} indicates a change in the size of the cells or their physiology. A permittivity curve 220 with two points and one dash shows a higher characteristic frequency f_{ch} in FIG. 9. The characteristic frequency f_{ch} can be determined from the inflection point of the curve 200 between the plateau region 204 and the plateau region 206.

The slope of the permittivity curve at the point of its characteristic frequency f_{ch} is a measure for the cell size distribution, with increasing slope indicating a more heterogeneous cell size distribution, and with flatter slopes of the permittivity curve 200 at the location of the characteristic frequency f_{ch} indicating more homogeneous cell size distributions.

In particular, the permittivity curves shown in FIG. 9 may be the curves of the real parts of the permittivity values determined.

In the exemplary embodiment of FIG. 9, f₁=50 kHz and f₂=20 MHz. Sensors according to exemplary embodiments of the invention permit a highly accurate determination of permittivity values over such a broad frequency range, whereby for many cell populations the β-dispersion region can be described very extensively. In particular, the sensor and the method for determining a value indicative of the impedance of a suspension according to exemplary embodiments of the invention are suitable for cell populations with a conductivity of a few 0.1 mS/cm to 100 mS/cm and with a permittivity of a few pF/cm to several hundred pF/cm.

An exemplary application for the sensor and the method for determining a value indicative of the impedance of a suspension according to exemplary embodiments of the invention are fermentation processes, for example in brewing beverages. However, the invention is generally broadly applicable for determining values indicative of the impedance of a suspension and for a downstream determination of permittivity values.

Although the invention has been described with reference to exemplary embodiments, it is apparent to those skilled in the art that various modifications may be made and equivalents used without departing from the scope of the invention. The invention is not intended to be limited by the specific embodiments described. Rather, it includes all embodiments covered by the appended claims.

1. A method for determining a value indicative of the impedance of a suspension in the framework of an impedance spectroscopy, comprising the following steps:
generating an excitation current through the suspension, the excitation current oscillating at an excitation frequency,
determining a first impedance measurement value on the basis of the excitation current and a first voltage at a first pair of measurement electrodes,
determining a second impedance measurement value on the basis of the excitation current and a second voltage at a second pair of measurement electrodes,
determining the value indicative of the impedance of the suspension by correlating the first impedance measurement value and the second impedance measurement value.

2. The method according to claim 1,
wherein said first pair of measurement electrodes comprises a first measurement electrode and a second measurement electrode, and wherein said second pair of measurement electrodes comprises said first measurement electrode and a third measurement electrode,
or
wherein the first pair of measurement electrodes comprises a first measurement electrode and a second measurement electrode, and wherein the second pair of measurement electrodes comprises a third measurement electrode and a fourth measurement electrode.

3. (canceled)

4. The method according to claim 1, wherein determining the value indicative of the impedance of the suspension comprises determining the difference between the first impedance measurement value and the second impedance measurement value, or comprises determining the difference between a first adjusted impedance value and a second adjusted impedance value, wherein the first adjusted impedance value and the second adjusted impedance value are obtained by applying a correction function to the first impedance measurement value and the second impedance measurement value, the correction function preferably representing the transmission behavior of the measurement arrangement.

5. The method according to claim 1, wherein determining the value indicative of the impedance of the suspension comprises determining the difference between a first geometry factor and a second geometry factor, wherein the first geometry factor represents the measurement geometry of the first pair of measurement electrodes and wherein the second geometry factor represents the measurement geometry of the second pair of measurement electrodes.

6. The method according to claim 1, wherein determining the value indicative of the impedance of the suspension is carried out according to the following formula:
$${Z = k \frac{1}{( {λ}_{1} - {λ}_{2 } )} {( {G}_{el}^{- 1} ( {Z}_{sig} ) \vert}_{1} - {G}_{el}^{- 1} ( {Z}_{sig} ) \vert}_{2} ) ,$$
wherein Z_{sig}|₁ denotes the first impedance measurement value, Z_{sig}|₂ denotes the second impedance measurement value, Gₑₗ⁻¹ denotes a correction function representing the transmission behavior of the measurement arrangement, λ₁ denotes a first geometry factor representing the measurement geometry of the first pair of measurement electrodes, λ₂ denotes a second geometry factor representing the measurement geometry of the second pair of measurement electrodes, and k denotes a proportionality constant.

7. The method according to claim 1, further comprising:
measuring the first voltage at the first pair of measurement electrodes, and measuring the second voltage at the second pair of measurement electrodes, wherein measuring the first voltage and measuring the second voltage are performed substantially simultaneously,
or
further comprising:
measuring the first voltage at the first pair of measurement electrodes, and measuring the second voltage at the second pair of measurement electrodes,
wherein measuring the first voltage and measuring the second voltage are performed in a time-shifted manner.

8-9. (canceled)

10. The method according to claim 1, wherein determining the first impedance measurement value and determining the second impedance measurement value comprises:
sampling the excitation current,
sampling the first voltage, and
sampling the second voltage
wherein the method further comprises the steps of:
setting a first sampling rate for sampling the excitation current,
setting a second sampling rate for sampling the first voltage, and
setting a third sampling rate for sampling the second voltage,
wherein the first sampling rate, the second sampling rate and the third sampling rate are set to at least 4 times the excitation frequency of the excitation current, in particular to substantially 4 times the excitation frequency of the excitation current.

11-12. (canceled)

13. The method according to claim 10, wherein the step of determining the first impedance measurement value comprises performing a first complex Fourier transform on the basis of the sampling values of the excitation current and the sampling values of the first voltage, and wherein the step of determining the second impedance measurement value comprises performing a second complex Fourier transform on the basis of the sampling values of the excitation current and the sampling values of the second voltage.

14. The method according to claim 1, further comprising:
determining a third impedance measurement value on the basis of the excitation current and a third voltage at a third pair of measurement electrodes, determining the value indicative of the impedance of the suspension by correlating the first impedance measurement value, the second impedance measurement value, and the third impedance measurement value.

15. The method according to claim 14,
wherein determining the value indicative of the impedance of the suspension comprises determining a first difference between the first impedance measurement value and the second impedance measurement value and determining a second difference between the first impedance measurement value and the third impedance measurement value and determining a third difference between the second impedance measurement value and the third impedance measurement value, or wherein determining the value indicative of the impedance of the suspension comprises determining a first difference between a first adjusted impedance value and a second adjusted impedance value and determining a second difference between the first adjusted impedance value and a third adjusted impedance value and determining a third difference between the second adjusted impedance value and the third adjusted impedance value, wherein the first adjusted impedance value, the second adjusted impedance value and the third adjusted impedance value are obtained by applying a correction function to the first impedance measurement value, the second impedance measurement value and the third impedance measurement value, the correction function preferably representing the transmission behavior of the measurement arrangement.

16. The method according to claim 14, wherein determining the value indicative of the impedance of the suspension comprises determining a first difference between a first geometry factor and a second geometry factor and determining a second difference between the first geometry factor and a third geometry factor and determining a third difference between the second geometry factor and the third geometry factor, wherein the first geometry factor represents the measurement geometry of the first pair of measurement electrodes, the second geometry factor represents the measurement geometry of the second pair of measurement electrodes, and the third geometry factor represents the measurement geometry of the third pair of measurement electrodes,
wherein determining the value indicative of the impedance of the suspension is carried out according to the following formula:
$${Z}^{2 } = {k}_{2} \frac{{{{λ}_{3} ( {G}_{el}^{- 1} ( {Z}_{sig} ) \vert}_{2} - {G}_{el}^{- 1} ( {Z}_{sig} ) \vert}_{1} )}{( {λ}_{1} - {λ}_{2} ) ( {λ}_{1} - {λ}_{3} ) ( {λ}_{2} - {λ}_{3} )} ++ {k}_{2} \frac{{{{λ}_{2} ( {G}_{el}^{- 1} ( {Z}_{sig} ) \vert}_{1} - {G}_{el}^{- 1} ( {Z}_{sig} ) \vert}_{3} )}{( {λ}_{1} - {λ}_{2} ) ( {λ}_{1} - {λ}_{3} ) ( {λ}_{2} - {λ}_{3} )} ++ {k}_{2} \frac{{{{λ}_{1} ( {G}_{el}^{- 1} ( {Z}_{sig} ) \vert}_{3} - {G}_{el}^{- 1} ( {Z}_{sig} ) \vert}_{2} )}{( {λ}_{1} - {λ}_{2} ) ( {λ}_{1} - {λ}_{3} ) ( {λ}_{2} - {λ}_{3} )} ,$$
wherein Z_{sig}|₁ denotes the first impedance measurement value, Z_{sig}|₂ denotes the second impedance measurement value, Z_{sig}|₃ denotes the third impedance measurement value, Gₑₗ⁻¹ denotes a correction function that represents the transmission behavior of the measurement arrangement, λ₁ denotes a first geometry factor that represents the measurement geometry of the first pair of measurement electrodes, λ₂ denotes a second geometry factor that represents the measurement geometry of the second pair of measurement electrodes, λ₃ denotes a third geometry factor that represents the measurement geometry of the third pair of measurement electrodes, and k2 denotes a proportionality constant.

17. (canceled)

18. A method for determining a value indicative of the impedance of a suspension in the framework of an impedance spectroscopy, comprising the following steps:
generating an excitation voltage, oscillating at an excitation frequency, applied to the suspension,
determining a first impedance measurement value on the basis of the excitation voltage and a first current through a first pair of measurement electrodes,
determining a second impedance measurement value on the basis of the excitation voltage and a second current through a second pair of measurement electrodes, determining the value indicative of the impedance of the suspension by correlating the first impedance measurement value and the second impedance measurement value.

19. A method for deriving at least one characteristic property of a suspension, comprising the steps of:
performing the method for determining a value indicative of the impedance of a suspension according to claim 1 a plurality of times, using a plurality of different excitation frequencies and determining a plurality of values indicative of the impedance of the suspension for the plurality of different excitation frequencies,
deriving a plurality of values indicative of the permittivity of the suspension based on the plurality of values indicative of the impedance of the suspension, and
deriving the at least one characteristic property of the suspension by correlating the plurality of values indicative of the permittivity of the suspension.

20. The method according to claim 19,
wherein said method for determining a value indicative of the impedance of a suspension is performed for between 2 and 50 different excitation frequencies, in particular for between 10 and 40 different excitation frequencies, further in particular for between 20 and 30 different excitation frequencies,
and/or
wherein the different excitation frequencies are from a frequency range from 100 kHz to 10 MHz, in particular from a frequency range from 50 kHz to 20 MHz.

21. (canceled)

22. The method according to claim 19,
wherein deriving the at least one characteristic property of the suspension includes generating a curve of the values indicative of the permittivity of the suspension over the different excitation frequencies,
and/or
wherein the suspension is a cell population and wherein the at least one characteristic property of the suspension comprises at least one property of number of living cells, size of the cells and homogeneity of the cells.

23. (canceled)

24. A sensor for determining a value indicative of the impedance of a suspension, comprising:
an oscillator circuit,
a pair of excitation electrodes coupled to the oscillator circuit, wherein an excitation current through the suspension, oscillating at an excitation frequency, can be generated across the pair of excitation electrodes by means of the oscillator circuit,
at least three measurement electrodes for measuring a first voltage in the suspension between a first pair of the at least three measurement electrodes and a second voltage in the suspension between a second pair of the at least three measurement electrodes, and
a data processing device configured to determine a first impedance measurement value on the basis of the excitation current and the first voltage, to determine a second impedance measurement value on the basis of the excitation current and the second voltage, and to determine the value indicative of the impedance of the suspension by correlating the first impedance measurement value and the second impedance measurement value.

25. The sensor according to claim 24,
wherein the at least three measurement electrodes are arranged between the pair of excitation electrodes.

26. (canceled)

27. The sensor according to claim 24,
wherein the at least three measurement electrodes are at least four measurement electrodes, wherein the first pair of the at least four measurement electrodes comprises a first measurement electrode and a second measurement electrode and wherein the second pair of the at least four measurement electrodes comprises a third measurement electrode and a fourth measurement electrode,
wherein the third and fourth measurement electrodes are arranged between the first and second measurement electrodes and/or wherein the third and fourth measurement electrodes are arranged on a different side of the sensor than the first and second measurement electrodes.

28-29. (canceled)

30. The sensor according to claim 24, wherein the data processing device is configured to determine the value indicative of the impedance of the suspension via determining the difference between the first impedance measurement value and the second impedance measurement value, or
wherein the data processing device is configured to determine the value indicative of the impedance of the suspension via determining the difference between a first adjusted impedance value and a second adjusted impedance value, wherein the data processing device is configured to determine the first adjusted impedance value and the second adjusted impedance value by applying a correction function to the first impedance measurement value and the second impedance measurement value, wherein the correction function preferably represents the transmission behavior of the measurement arrangement.

31. The sensor according to claim 24, wherein the data processing device is configured to determine the value indicative of the impedance of the suspension via determining the difference between a first geometry factor and a second geometry factor, wherein the first geometry factor represents the measurement geometry of the first pair of the at least three measurement electrodes and wherein the second geometry factor represents the measurement geometry of the second pair of the at least three measurement electrodes,
wherein the data processing device is configured to determine the value indicative of the impedance of the suspension according to the following formula:
$${Z = k \frac{1}{( {λ}_{1} - {λ}_{2 } )} {( {G}_{el}^{- 1} ( {Z}_{sig} ) \vert}_{1} - {G}_{el}^{- 1} ( {Z}_{sig} ) \vert}_{2} ) ,$$
wherein Z_{sig}|₁ denotes the first impedance measurement value, Z_{sig}|₂ denotes the second impedance measurement value, Gₑₗ⁻¹ denotes a correction function representing the transmission behavior of the measurement arrangement, λ₁ denotes a first geometry factor representing the measurement geometry of the first pair of the at least three measurement electrodes, λ₂ denotes a second geometry factor representing the measurement geometry of the second pair of the at least three measurement electrodes, and k denotes a proportionality constant.

32-39. (canceled)

40. The sensor according to claim 24, wherein the oscillator circuit is coupled to the pair of excitation electrodes via a transformer, wherein the transformer in particular has a parallel capacitance of 0.5 to 10 pF.

41-42. (canceled)

43. A sensor for determining a value indicative of the impedance of a suspension, comprising:
an oscillator circuit,
a pair of excitation electrodes coupled to the oscillator circuit, wherein an excitation voltage, oscillating at an excitation frequency, applied to the suspension can be generated across the pair of excitation electrodes by means of the oscillator circuit,
at least three measurement electrodes for measuring a first current in the suspension between a first pair of the at least three measurement electrodes and a second current in the suspension between a second pair of the at least three measurement electrodes, and
a data processing device configured to determine a first impedance measurement value on the basis of the excitation voltage and the first current, to determine a second impedance measurement value on the basis of the excitation voltage and the second current, and to determine the value indicative of the impedance of the suspension by correlating the first impedance measurement value and the second impedance measurement value.

44. A computer program comprising program instructions which, when executed on a data processing system, perform a method according to claim 1.
